(19) United States
(12) Patent Application Publication
(10) Pub. No.: US 2024/0207508 A1
(43) Pub. Date: Jun. 27, 2024

(54) HYBRID BIOELECTRONIC/ENGINEERED CELL IMPLANTABLE SYSTEM FOR THERAPEUTIC AGENTS DELIVERY AND APPLICATIONS THEREOF

(71) Applicants: NORTHWESTERN UNIVERSITY, Evanston, IL (US); WILLIAM MARSH RICE UNIVERSITY, Houston, TX (US)

(72) Inventors: Jonathan Rivnay, Chicago, IL (US); Omid Veiseh, Bellaire, TX (US); Jacob Robinson, Houston, TX (US); Samantha T. Fleury, Houston, TX (US); Matthew C. Parker, Houston, TX (US); Fatima T. Alrashdan, Houston, TX (US)

(21) Appl. No.: 18/287,671

(22) PCT Filed: Apr. 21, 2022
(86) PCT No.: PCT/US2022/025686

§ 371 Date: Oct. 20, 2023

Related U.S. Application Data
(60) Provisional application No. 63/177,806, filed on Apr. 21, 2021.

(51) Int. Cl. A61M 5/142; A61M 5/172; G16H 40/67

(52) U.S. Cl. CPC A61M 5/14276; A61M 5/1723; G16H 40/67; A61M 2205/3306

(57) ABSTRACT

A bioelectronic implantable device includes engineered cells and an electronically controlled stimulator that regulates a quantity and timing of therapeutic agent produced by the engineered cells.

### CROSS-REFERENCE TO RELATED PATENT APPLICATIONS

This application claims priority to and the benefit of U.S. Provisional Patent Application Ser. No. 63/177,806, filed Apr. 21, 2021, which is incorporated herein in its entirety by reference.

This application is also related to co-pending PCT patent applications, entitled “Engineered Cells for Producing Therapeutic Agents to be Delivered by a Hybrid Bioelectronic Device”, with Attorney Docket No. 0116936.266WO23, and “Hybrid Bioelectronic/Engineered Cell Wearable System For Therapeutic Agents Delivery And Applications Thereof”, with Attorney Docket No. 0116936.266WO22, respectively, which are filed on the same day that this application is filed, and with the same applicant as that of this application, which are incorporated herein by reference in its entirety.

### STATEMENT AS TO RIGHTS UNDER FEDERALLY-SPONSORED RESEARCH

This invention was made with government support under FA8650-21-2-7119 awarded by the Air Force Research Laboratory. The government has certain rights in the invention.

### FIELD OF THE INVENTION

The present disclosure relates generally to the field of biomedical engineering, and more particularly to hybrid bioelectronics/engineered cell systems for delivery of therapeutic agents produced by genetically engineered cells into an individual's body, and applications of the same.

### BACKGROUND OF THE INVENTION

The background description provided herein is for the purpose of generally presenting the context of the present invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

The function of implanted cells, tissues, and devices depends on numerous factors, including the ability to provide a product and the biological immune response pathway of the recipient (Anderson et al., Semin Immunol (2008) 20:86-100; Langer, Adv Mater (2009) 21:3235-3236). A stable environment and precise control is necessary and desired by the implanted cells, tissues, and devices for the purpose of producing and delivering any therapeutic agents to the recipient.

Therefore, there remains an imperative need for a system to protect cells from the host, facilitate improved delivery, viability, potency, and remote control of activation so as to impart a beneficial effect on the fidelity and function of implanted cells, tissues, and devices.

### SUMMARY OF THE INVENTION

In light of the foregoing, this invention discloses a bioelectronic system having (i) implanted biohybrid (bioelectronic/engineered cell) device, and (ii) wearable external hub, which, in concern provide precise (timing and dose) delivery of biomolecules synthesized by genetically engineered cells directly into the blood stream. Sensors both on-board the implantable, in the external hub, or coupled to other commercial off the shelf wearables sensors, provide input for dose delivery timing, depending on the application. The implantable device includes engineered mammalian cells that are genetically modified to deliver the biomolecule of interest, and to do so upon optoelectronic trigger. These cells are controlled by a series of LEDs and photodiodes and are supported via optoelectronic oxygen generation within an encapsulated, immuno-isolating cell-housing compartment. The implant includes multiple of the same type of cell in different compartments and/or other engineered cells for multi-molecule delivery. The implant also includes sensors, power management, and communication on a small form factor footprint. This work establishes a generalizable engineering framework for hybrid bioelectronic/engineered cell implantable system for therapeutic agents precise delivery.

In one aspect of the invention, a hybrid bioelectronic implantable device containing engineered cells for delivery of therapeutic agents to a subject, the device comprising an implantable device implantable inside the body of the subject, wherein the implantable device comprises at least one cell housing containing at least one type of the engineered cells, wherein each of the engineered cells contains an optogenetic system; an optical stimulating system within the at least one cell housing, wherein the optical stimulating system has at least one light source, wherein the optogenetic system is configured to receive a signal light from the at least one light source to control production of at least one type of therapeutic agent and a reporter agent by the engineered cells; a permeable encapsulation material on at least a portion of a surface of the implantable device; and an external hub disposable outside of the body of the subject, wherein in use, the external hub and the implantable device are positioned in communication via a communication method using at least one of radio frequency (RF), near field communication (NFC), magnetoelectric (ME), and ultrasound; wherein in use, the at least one type of therapeutic agent is released from the cell housing into the subject's body through the permeable encapsulation.

In one embodiment, the device further comprising a controller in communication with the optical stimulating system and the sensing system, wherein the controller is configure to control the production of the at least one type of therapeutic agent according to a control algorithm.

In one embodiment, the engineer cells are configured to start the production of the at least one type of therapeutic agent when the optogenetic systems of the engineered cells receive a signal light having a first wavelength from the optical stimulating system.

In one embodiment, the device further comprising a sensing system within the at least one cell housing, sensing a fluorescent light or bioluminescence generated by the reporter agent, the engineer cells are configured to stop the production of the at least one type of therapeutic agent when either the the optogenetic systems of the engineered cells receive a signal light having a second wavelength, or the sensing system detects a predetermined level of the fluorescent light or bioluminance generated by the reporter agent.

In one embodiment, a ratio of the amount of the produced reporter agent to the amount of the produced at least one type of therapeutic agent is fixed.

In one aspect of the invention, a hybrid bioelectronic implantable device containing engineered cells for delivery of therapeutic agents to a subject, the device comprising an implantable device implantable inside the subject's body, wherein the implantable device comprises at least one cell housing containing the engineered cells; and an optical stimulating system within the at least one cell housing, wherein the optical stimulating system is configured to control production of at least one type of therapeutic agents by the engineered cells.

In one embodiment, the optical stimulating system comprises a light source to generate a light of first wavelength and a light of second wavelength different from the light of first wavelength.

In one embodiment, each of the engineered cells contains an optogenetic system configured to receive the light generated by the light source of the optical stimulating system.

In one embodiment, the engineered cells are configured to start producing the at least one type of therapeutic agent when the optogenetic systems in the engineered cells receive the light of first wavelength.

In one embodiment, the engineered cells are configured to stop producing the at least one type of therapeutic agent when the optogenetic systems in the engineered cells receive the light of second wavelength.

In one embodiment, the implantable device further comprises a sensing system disposed in the at least one cell housing, wherein the sensing system detects a signal generated by a reporter agent produced by the engineered cells, and wherein the signal comprises at least one of fluorescent light signal, bioluminescence signal, impedance signal, pigment signal, and free radical signal.

In one embodiment, the engineered cells stop producing the at least one type of therapeutic agent when the signal detected by the sensing system reaches to a predetermined level.

In one embodiment, the sensing system comprises a photodiode.

In one embodiment, the reporter agent and the at least one type of therapeutic agent are produced at a fix ratio.

In one embodiment, the implantable device further comprises a permeable encapsulation material on at least a portion of its surface to allow the at least one type of therapeutic agent to be released into the subject's body through the permeable encapsulation.

In one embodiment, the permeableencapsulation material comprises a multi-layer membrane.

In one embodiment, the multi-layer membrane comprises a first layer having sub-micron size pores configured to prevent immune cells of the subject from passing through the multi-layer membrane, and a second layer having micron-size pores configured to enhance vascularization.

In one embodiment, when in use, the implantable device is wirelessly coupled to an external hub disposed outside of the subject's body.

In one embodiment, the hybrid bioelectronic implantable device and the external hub are in communication with each other.

In one embodiment, the external hub is configured to collect at least one external parameter or biometric parameter, and wherein the external parameter or biometric parameter comprises an environment temperature, a location of the subject, a body temperature, a blood pressure, a heart rate, and a speed of the subject.

In one embodiment, the device further comprising a control unit in communication with the stimulating system and the sensing system to control the stimulating system and the sensing system; and a memory unit in communication with the control unit.

In one embodiment, the memory unit is configured to store a control algorithm to regulate production of the at least one type of therapeutic agent.

In one embodiment, the control unit and memory unit locate in the external hub.

In one embodiment, the communication between the hybrid bioelectronic implantable device and the external hub is via a communication method using at least one of radio frequency (RF), near field communication (NFC), magnetoelectric (ME), and ultrasound.

In one embodiment, the device further comprising a battery that is in power communication with the external hub.

In one embodiment, the implantable device comprises an oxygen generator producing oxygen for the engineered cells.

In one embodiment, the engineered cells comprise a first type of the engineered cells producing a first type of the therapeutic agent, and a second type of the engineered cells producing a second type of therapeutic agent.

In one embodiment, the at least one cell housing comprises a first cell housing and a second cell housing, and wherein the first cell house contains the first type of the engineered cells, and the second cell housing contains the second type of the engineered cells, respectively.

In one embodiment, the implantable device is implantable subcutaneously, pericardially, intracranially, or intraperitoneally.

In one embodiment, the photodiode is enclosed in a photodiode encapsulation, wherein the photodiode encapsulation reduces the amount of the light of the first wavelength reaching the photodiode.

In another aspect of the invention, a method for delivering at least one type of therapeutic agent to a subject by a hybrid bioelectronic implantable device, the method comprising controlling, by a control unit, a light source of an optical stimulating system located in a first cell housing of an implantable device to produce a light of first wavelength, wherein the first cell housing contains a first type of engineered cells having an optogenetic system in each of the engineered cells; illuminating the first type of engineered cells with the light of first wavelength for a illumination time period; and starting production of a first type of therapeutic agent by the engineered cells when the optogenetic systems in the engineered cells receive the light of first wavelength.

In one embodiment, the device further comprising controlling, by the control unit, the light source of the optical stimulating system located in the first cell housing of the implantable device to produce a light of second wavelength, wherein the second wavelength is different from the first wavelength; illuminating the first type of engineered cells with the light of second wavelength; and stopping the production of the first type of therapeutic agent by the engineered cells when the optogenetic systems in the engineered cells receive the light of second wavelength.

In one embodiment, the time interval between the production of the light of first wavelength and the production of the light of the second wavelength is controlled by the control unit according to a control algorithm.

In one embodiment, the production of the first type of therapeutic agent by the engineered cells last for a production time period longer than the illumination time period.

In one embodiment, the method further comprising producing a reporting agent by the engineered cells when the optogenetic systems in the engineered cells receive the light of first wavelength.

In one embodiment, the reporter agent generates a signal of fluorescent light signal, bioluminescence signal, impedance signal, pigment signal, or free radical signal.

In one embodiment, the method further comprising stopping the production of the first type of therapeutic agent by the engineered cells when the signal generated by the reporter agents detected by a sensing system reaches a predetermined level, wherein the sensing system locates inside the first cell housing.

In one embodiment, a ratio of the amount of the produced reporter agent to the amount of the produced first type of therapeutic agent is fixed.

In one embodiment, the implantable device comprises a second cell housing containing a second type of the engineered cells, wherein the method further comprises controlling, by the control unit, the optical stimulating system located in the second cell housing to produce the light of first wavelength, illuminating the second type of the engineered cells with the light of first wavelength; and starting production of a second type of therapeutic agent by the second type of engineered cells when the optogenetic systems in the second type of engineered cells receive the light of first wavelength.

In one embodiment, the first type of therapeutic agent is different from the second types of therapeutic agent.

In one embodiment, the hybrid bioelectronic implantable device further comprises an external hub.

In one embodiment, the external hub is in communication with the implantable device via at least one communication method using radio frequency (RF), near field communication (NFC), magnetoelectric (ME), and ultrasound.

In one embodiment, the method further comprising power charging the implantable device with the external hub wirelessly.

In one embodiment, the method further comprising power charging the implantable device with an on-board battery.

In one embodiment, the hybrid bioelectronic implantable device further comprising an on-board battery providing power to the implantable device.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 depicts phases of peripheral and central clocks in response to an 8 hr shift, for normal entrainment (left), providing therapy affecting only the central clock (middle), and the proposed NTRAIN approach (right) with therapy targeting both central and peripheral clocks in accordance with an illustrative embodiment.

FIG. 2 is a table that depicts the rationale for using optical induction to perform control and feedback in accordance with an illustrative embodiment.

FIGS. 3A-3D provide different embodiments of hybrid bioelectronic device. FIG. 3A illustrates an implantable embodiment having a single cell housing containing engineered cells.

FIG. 3B illustrates an implantable embodiment having plurality of cell housings containing same or different engineered cells. FIG. 3C illustrates an implantable embodiment having one or more cell housings integrated with power transduction management system, optoelectronics and other accessary systems e.g., O₂ generation system. FIG. 3D illustrates a wearable embodiment having media cartridge separate from the cell housing.

FIG. 4 depicts operations performed to implement the proposed NTRAIN system in accordance with an illustrative embodiment.

FIG. 5 is a graphical depiction of the proposed synthetic biology circuit for optogenetic control of the peptide therapeutic Orexin A in accordance with an illustrative embodiment.

FIG. 6 depicts preliminary data showing that ARPE-19 cells can be made to express luciferase with high on/off ratio in response to blue light using an EL222 optogenetic system in accordance with an illustrative embodiment.

FIG. 7 shows a biohybrid precision control scheme based on co-production of therapeutic peptide and proxy reporter fluorophore (GFP*) in accordance with an illustrative embodiment.

FIG. 8 shows a comparison of traditional optogenetic control strategies that use constant illumination to activate the ion channels to the proposed step-function opsin control strategy that utilizes a blue LED to open the light-gated channels and an orange LED to close the channels in accordance with an illustrative embodiment.

FIGS. 9A-B show wired and wireless prototype devices. FIG. 9A shows design of the top side of the circuit board with the LEDs and photodiodes. FIG. 9B shows wired prototype device with the wires extending out the right side of the device.

FIG. 10 shows charts regarding utilization of blue LEDs and photodiodes to measure green fluorescence of cells.

FIG. 11 shows top view of blue and orange LEDs on an encapsulated circuit board at the end of the soak test.

FIGS. 12A-C show a filter and its application to isolate the photodiode from the blue LED. FIG. 12A shows a chart of optical density of the Wratten 12 filter. FIG. 12B shows the laser cut optical filter is bent into a box shape before being glued to the photodiode. FIG. 12C shows a chart of photovoltages obtained with and without the optical filter.

FIGS. 13A-B show device molds. FIG. 13A shows an aluminum device mold. FIG. 13B shows a PDMS device molded by the aluminum mold.

FIGS. 14A-D show power transmission elements and output power through porcine tissue. FIG. 14A shows battery-powered transmitter. FIG. 14B shows ME film used as a receiver.

FIG. 14C shows the setup for testing power transmission. FIG. 14D shows a chart of output power transferred through 2 cm porcine tissue.

FIGS. 15A-B show synthesis reaction for RZA15. FIG. 15A shows a process for RZA15 synthesis. FIG. 15B shows NMR and ES MS characterization of the resulting RZA15 product.

FIGS. 16A-B show synthesis reaction for RZA15 UPVLVG. FIG. 16A shows systhesis process; FIG. 16B shows NMR and elemental characterization of the product.

FIG. 17 shows an aid device for loading the bioelectric device with cells.

FIG. 18 is a block diagram of a computing system to perform operations described herein in accordance with an illustrative embodiment.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used herein, the term “and/or” includes any and all combinations of one or more of the associated listed items.

As used in the disclosure, the term “implantable” refers to an ability of a device to be positioned at a location within a body of a user, such as subcutaneously, within a body cavity, or etc. Furthermore, the terms “implantation” and “implanted” refer to the positioning of a device at a location within a body of a user, such as subcutaneously, within a body cavity, or etc.

As used in the disclosure, the term “wearable” refers to articles, adornments or items designed to be worn by a user, incorporated into another item worn by a user, act as an orthosis for the user, or interfacing with the contours of a user's body.

As used in the disclosure, “biocompatible” material is a material that is compatible with living tissue or a living system by not being toxic or injurious and not causing immunological rejection.

As used in the disclosure, “therapeutic agent” refers to any substance that provides therapeutic effects to a disease or symptom related thereto. In certain embodiments, a therapeutic agent refers to a substance that provides therapeutic effects to any diseases or biological or physiological responses to the diseases.

As used in the disclosure, the term “therapy” refers to any protocol, method, and/or agent that can be used in the management, treatment, and/or amelioration of a given disease, or a symptom related thereto. In certain embodiments, the terms “therapies” and “therapy” refer to a biological therapy, supportive therapy, and/or other therapies known to one of skill in the art, such as medical personnel, useful in the management or treatment of a given disease, or symptom related thereto.

As used in the disclosure, “treat”, “treatment”, and “treating” refer to the reduction or amelioration of the progression, severity, and/or duration of a given disease resulting from the administration of one or more therapies (including, but not limited to, the administration of microspheres disclosed herein). In certain embodiments, the terms refer to the reduction of pain associated with one or more diseases or conditions.

As used in the disclosure, “engineered cell(s)” refers herein to cells having been engineered, e.g., by the introduction of an exogenous nucleic acid sequence or specific alteration of an endogenous gene sequence. An exogenous nucleic acid sequence that is introduced may comprise a wild type sequence of any species that may be modified. An engineered cell may comprise genetic modifications such as one or more mutations, insertions and/or deletions in an endogenous gene and/or insertion of an exogenous nucleic acid (e.g., a genetic construct) in the genome. An engineered cell may refer to a cell in isolation or in culture. Engineered cells may be “transduced cells” wherein the cells have been infected with e.g., an engineered virus. For example, a retroviral vector may be used, such as described in the examples, but other suitable viral vectors may also be contemplated such as lentiviruses. Non-viral methods may also be used, such as transfections or electroporation of DNA vectors. DNA vectors that may be used are transposon vectors. Engineered cells may thus also be “stably transfected cells” or “transiently transfected cells”. Transfection refers to non-viral methods to transfer DNA (or RNA) to cells such that a gene is expressed. Transfection methods are widely known in the art, such as calcium phosphate transfection, PEG transfection, and liposomal or lipoplex transfection of nucleic acids. Such a transfection may be transient, but may also be a stable transfection wherein cells can be selected that have the gene construct integrated in their genome.

Present system described herein features an implantable device housing engineered cells, e.g., ARPE-19 cells, that produce or are capable of producing one or more therapeutic agents. The therapeutic agent may be a biological substance, such as a nucleic acid (e.g., a nucleotide, DNA, or RNA), a polypeptide, a lipid, a sugar (e.g., a monosaccharide, disaccharide, oligosaccharide, or polysaccharide), a small molecule, etc. In some embodiments, the therapeutic agent is a polypeptide. Each engineered cell comprises a promoter operably linked to a nucleotide sequence encoding the polypeptide. In such an implementation, the promoter can essentially be a nucleotide sequence. In some embodiments, the therapeutic agent is a replacement therapy or a replacement protein, e.g., useful for the treatment of a blood clotting disorder or a lysosomal storage disease in a subject.

In some embodiments, the implantable device includes one or more engineered cells, which can be provided as a cluster or disposed in a microcarrier. In some embodiments, the engineered cells produce or release a therapeutic agent (e.g., a polypeptide) for at least 0.5 day, 1 day, 10 days, or more, when implanted into a subject. In one embodiment, more than one therapeutic agent are produced by the engineered cells. In one embodiment, the implantable device may include one or more types of engineered cells, one type of the engineered cells may produce a therapeutic agent which is different from the therapeutic agent produced by other types of the engineered cells.

In another aspect, the present disclosure features a method of treating a subject comprising administering to the subject an implantable device housing the engineered cells producing at least one therapeutic agents.

In one illustrative embodiment, the subject is a human and the engineered active cell is a human cell. Alternatively, the subject may be a dog, cat, or other animal. In some embodiments, the therapeutic agent produced by the engineered cell(s) is a replacement therapy or a replacement protein, e.g., useful for the treatment of metabolic diseases. In some embodiments, the implantable device is formulated for implantation or injection into a subject.

The produced therapeutic agents can be evaluated by an art-recognized reference method, e.g., polymerase chain reaction or in situ hybridization for nucleic acids; mass spectroscopy for lipid, sugar and small molecules; microscopy and other imaging techniques for agents modified with a fluorescent or luminescent tag, and ELISA or Western blotting for polypeptides. In some embodiments, the implantable device comprises an encapsulating component (e.g., formed in situ on or surrounding the engineered cells, or preformed prior to combination with the engineered cells). In other embodiments, the implantable device is chemically modified, as described herein.

Thus, described herein is a hybrid bioelectronics/engineered cells pharmacy that enables the production of therapeutic agents within the subject. The therapeutic agents can be used to control pain, treat metabolic disorders, treat immune system disorders, treat psychiatric disorders, improve fertility, and any other medical or health conditions requiring a frequent and/or precise administration of therapeutic agents.

The present invention provides a therapy having a timing and dosing control which far exceeds the existing therapies and/or bioelectronics. The proposed system is able to achieve 1) specific biological action on select target receptors or molecules that cannot be accomplished with current bioelectronics, and 2) precise control of timing and dosage that cannot be accomplished with current synthetic biology.

In this embodiment, the living pharmacy includes engineered cells that produce therapeutic peptides with a timing and dose profile that is tightly controlled by optical triggers from an implanted bioelectronic carrier device (i.e., implant device). Together with a subcutaneous bioelectronic carrier, the proposed system overcomes the major challenges facing hybrid bioelectronic devices, including: 1) selective activity on biological targets, 2) precise control of biomolecule production, 3) high dose to load volume ratio, 4) protection from the host's immune response, and 5) wireless data and power transfer through biological tissue. In alternative implementations, the system may have fewer, additional, and/or different features.

The biohybrid system of the present invention provides a general platform for precise drug delivery and regulation that can be implanted long-term to treat short term or long term diseases, physical and/or mental health conditions, as well as improve user health and performance, without the need to carry pharmaceuticals. In one exemplary embodiment discussed below, the proposed system minimizes the adverse health consequences of circadian misalignment by achieving at least a 50% reduction in entrainment time using an implanted living biohybrid pharmacy that remains functional for an extended period of time (e.g., 30 days, 60 days, 90 days, etc.). In other embodiments, the disclosed invention provides treatments to diseases and/or physiological conditions including metabolic diseases, e.g., obesity and diabetes (e.g., Type 1, Type 2) by producing metabolically active molecules, e.g., leptin, ACTH, insulin, and GLP-1; cancers by producing therapeutic cytokines e.g., IL-2, IL-12, IL-15, GCSF; autoimmune diseases by producing regulated molecules e.g., IL-10, IL-35, treatment resistant depression and pains by producing neuropeptides e.g., GLYX-13, rapastinel, and ziconotide; osteoporosis by producing PTH; infertility by producing gonadotropin releasing hormone GnRH; and etc.

To achieve these metrics, the system focuses on five main innovations to overcome barriers of current bioelectronic and synthetic biology technologies, as well as an innovative approach to accelerating entrainment. These innovations, which are described in detail below, include performing selective activity on biological targets using natural peptides, precisely controlling biomolecule production, obtaining a high dose to load volume ratio, providing protection from the host's immune response, and wirelessly transmitting data and power through biological tissue.

With respect to selective activity on biological targets using natural peptides, the inventors have proposed using engineered allogeneic cells to produce select peptides that are otherwise naturally produced by the body to control pain, fight disease, regulate sleep cycles, treat metabolic related conditions, and etc. It is to be understood that in other applications the system can be used to produce other types of therapeutic agents. The body naturally produces these native peptides which are structurally similar to their recombinant counterparts. However, the native peptides diverge in potency and bioactivity. Significantly, it is noted that native peptides have not been commercialized due to their instability. However, the inventors have determined that a cell delivery platform which supports on demand in situ production use of native peptides as therapeutics is feasible. These naturally produced molecules are excellent candidates to regulate the specific activity of central and peripheral circadian clocks because they act selectively on these biological targets and do not elicit the immune responses that are shortcomings of recombinant peptides or exogenous drugs (i.e., antidrug antibodies). Furthermore, these peptides feature short metabolic half-lives, and are useful for relatively fast cessation of drug production. These traits make the proposed cell platform uniquely suited to deliver such biologics on demand. Additionally, it has been demonstrated that allogeneic cells encapsulated and implanted in vivo can survive for greater than 130 days in non-human primates without immunosuppression, suggesting that the proposed solution can enable living engineered cells-based devices with lifetimes that can extend for several months, or even years.

The novel system is also able to perform precision dosing with closed-loop bioelectronic control. A key challenge for biological production of therapeutic agents is controlling the production levels, which can vary due to cell health, temperature, and metabolism. To overcome this challenge, the proposed system includes a state-of-the art bioelectronic feedback control system based on optogenetically controlled therapy production and fluorescent tracking of therapy production levels. In alternative embodiments, the feedback control system may not be used. Cells engineered with optogenetic systems start protein production in response to exposure to specific activating light signals. By controlling light exposure, production of therapeutic agents can be controlled.

Another innovation of the proposed system is a bioelectronic feedback loop based on fluorescent tracking of the production levels. To create this feedback control loop, the cells are engineered to produce a fluorescent protein at a fixed ratio relative to the therapeutic agents. Using this fluorescence measurement as the feedback signal, the system is able to regulate the on time of the engineered cells to maintain a stable fixed point of therapeutic agents production with precision that exceeds synthetic biological feedback loops.

In some embodiments, in addition to the fluorescent signal provided by the fluorescent proteins, the bioelectronic feedback loop is based on biochemical signals which can be electronically detected. In addition to the fluorescent signal, the biochemical signals may include bioluminescence signal, impedance signal, pigment signal, and free radical signal.

In some embodiments, the proposed system also provides high-dose to load volume with on-chip life support and engineered cells. To support a higher concentration of therapeutic agents produced by the implanted device, one could increase the density of the engineered cells inside the chassis. However, the maximum cell density is currently limited by the amount of diffuse oxygen available in the subcutaneous space. To reach higher cell densities, the carrier is engineered to produce local O₂ with the bioelectronic carrier. Furthermore, using synthetic biology tools, the system amplifies transcription of the therapeutic peptides and programs cells to be resilient to senescence and cell death.

In another aspect of the invention, the proposed system also provides protection from the host immune response using a small molecule coating. Specifically, engineered cells are encapsulated within a life support system that protects them from the immune system of the host and that supports cell viability and productivity. Hydrogels and permeable or semi-permeable membrane biomaterials can be used to block cells from the body's immune system via their physical, hierarchical pore structure and biochemical functionalization. The hydrogels and permeable or semi-permeable membrane biomaterials further promote vascularization near the device/tissue interface to boost oxygenation from the body's circulatory system.

In some embodiments, the proposed system also performs efficient wireless data and power transfer through tissue using magnetoelectrics. Traditional wireless power delivery by electromagnetic or ultrasound waves has to overcome absorption by tissue and impedance mismatches between air, bone, and tissue, and such techniques often struggle to provide large powers to small bioelectronic devices. In contrast, magnetic fields are not affected by tissue-absorption or differences in interfacial impedances. The proposed approach uses recently-developed magnetoelectric (ME) technology and custom low power application specific integrated circuits (ASICs) to enable compact, reliable, wireless transmission of power and data, providing superior power densities and alignment tolerance. This approach for wireless power can enable ultra-miniature versions of the implantable device that can be injectable. Alternatively, the proposed system can use other sources of power and data transfer such as inductive coupling, photovoltaic data and power control, radio frequency (RF) data and power control, inductive data and power control, ultrasound data and power control, direct current (DC) coupled data and power control, etc. In alternative implementations, battery power or energy harvesting from the body could reduce or eliminate the need for wireless power. The system can be used for delivery of single or multiple therapeutics. In one embodiment, the engineered cells produce one or more therapeutic agents. In another embodiment, there are more than one type of engineered cells housed either in one cell housing, or each type of the engineered cells is housed in separated housings. In one exemplary embodiment of multiple therapeutic delivery, multi-clock targeting with precision timing for circadian rhythm regulation can be performed by the proposed system, as discussed below.

Unlike bioelectronic or gated biofluidic systems that feature pre-filled (or even refillable) reservoirs of drugs, the proposed system delivers naturally-occurring peptides throughout its functional lifetime without the need to stock, carry, or refill therapies that are vulnerable to loss, degradation, or that add to the already burdensome load carried by the user. The developed technology will serve as a platform whereby the optical control and feedback to achieve precision therapies can be applied to delivery of a broad swath of naturally occurring peptides/proteins by following the procedures and protocols described herein.

Thus, the disclosed system provides a hybrid bioelectronics platform and forms the basis and components for a number of bioelectronic and biohybrid tools to address or alleviate dysfunction and injury, to enhance readiness and performance, to treat pain, to treat disease, improves metabolism, and etc. The rationale behind the proposed system, along with details of its implementation, use, and testing are described in more detail below.

### Biohybrid System For On Demand Therapy

Engineering a cell-based hybrid bioelectronic system for on demand therapy is a challenge involving a careful balance between cellular and bioelectronic device engineering. Several factors have to be taken into consideration to balance the strengths of each pillar and to minimize their drawbacks and deficiencies. Cells possess their own natural machinery to synthesize and release specific biomolecules. Furthermore, their machinery can be hacked to externally induce such production, but without the level of timing, control, and user interfacing that is possible with bioelectronics. On the other hand, optoelectronic components have inherent chemical and mechanical mismatches with cells and tissue that can limit their lifetime (degradation, rejection), or make them otherwise incompatible with reliable use in vivo. Striking the proper balance between the two is important.

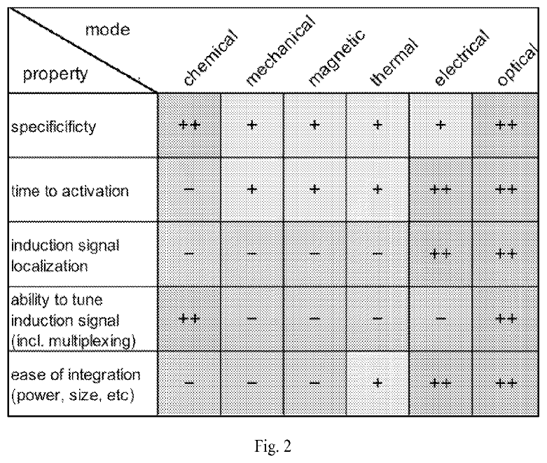

Key design decisions that permeate the proposed system focus on (i) promoting long-term viability/efficiency and (ii) controlling and creating feedback-loop of therapeutic agents production. To promote viability of engineered cells, the proposed system both genetically engineers cells to be more resilient, and in some embodiments can also produce O₂ to support them. For control and feedback-loop, optical induction can be used. Compared to other cellular control mechanisms, optical induction methods enable fast response, tunable, localized induction properties (wavelength control), and are readily integrated into the platform with minimal power and size demands. Furthermore, optoelectronic cell interfacing enables innovative precision low-power dosing control. FIG. 2 is a table that depicts the rationale for using optical induction to perform control and feedback in accordance with an illustrative embodiment.

### Technological Components of the System

In an illustrative embodiment, the proposed system includes an implantable device, (e.g., subcutaneous implanted) featuring individually-controlled cell housing, and an external wearable hub (extHub) (hardware and software) for power, user interface, and sensing. In alternative implementations, only the implantable device may be used. It should be noted that, in the application, the term “cell house”, “cell housing” and “cell well” are used interchangeably.

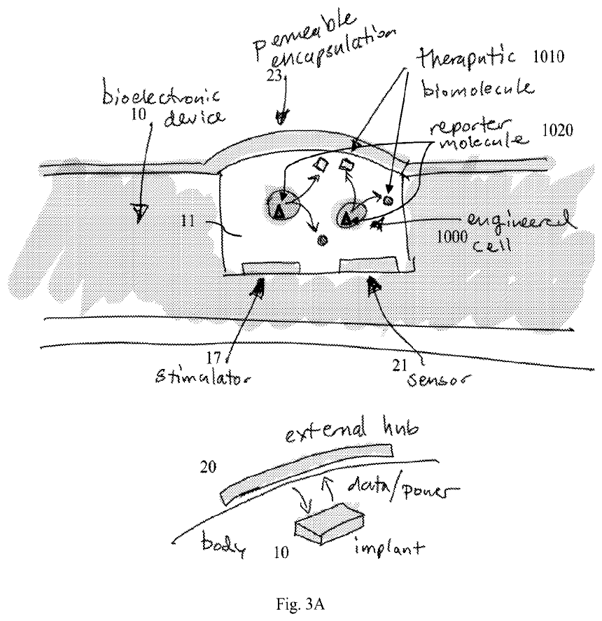

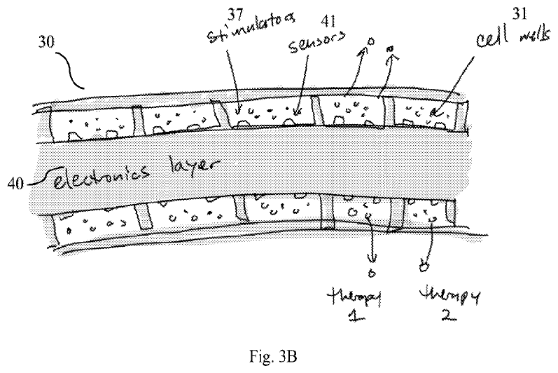

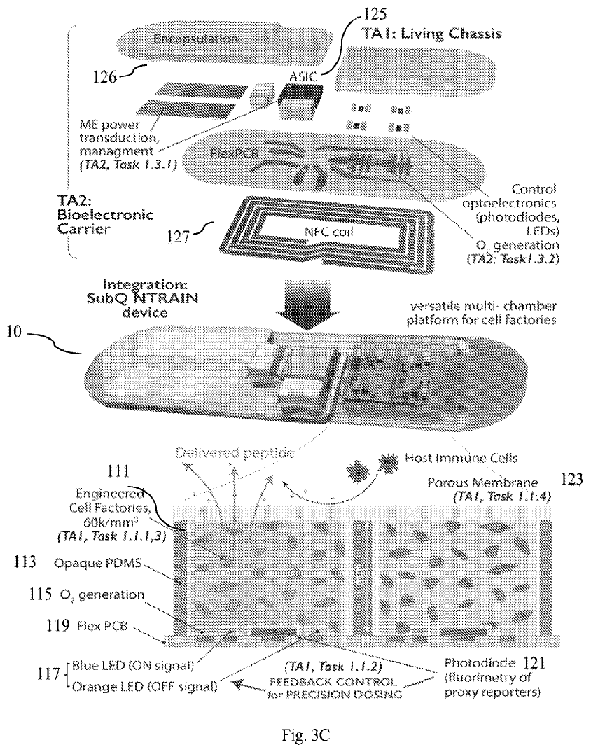

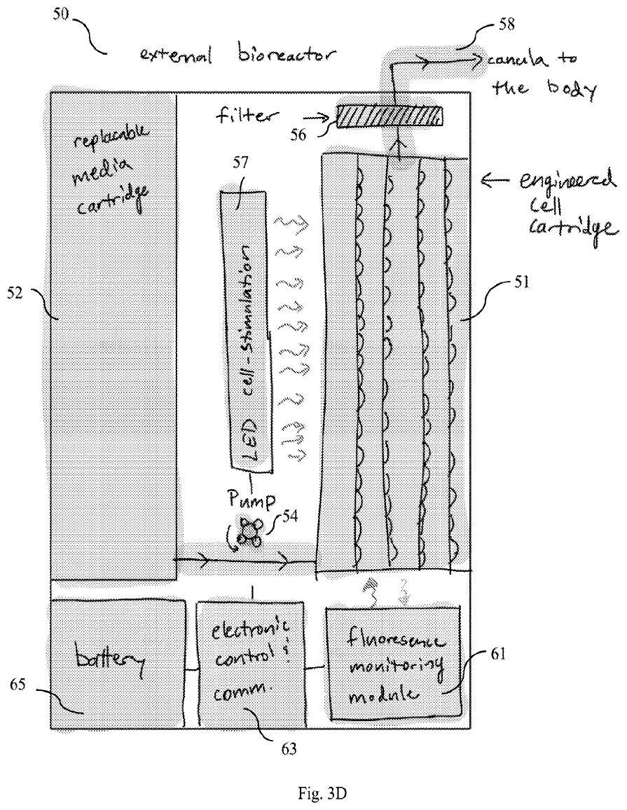

FIG. 3A provides an illustrative diagram showing the general structure of a unit of the bioelectronics device. As shown in the lower panel, the bioelectronics may comprising a unit of the implantable device 10 inside the body of a user, and an external hub 20 located outside the body of the user. The external hub 20 is in communication with the implantable device 10 for power charging and data exchanging/transmission. As shown in the upper panel, the implantable device 10 comprising a cell housing 11 for containing engineered cells 1000 which produces the therapeutic agents 1010 and a reporter agent/molecule 1020. A stimulator 17 for triggering the production of the therapeutic agents 1010 and the reporter agent/molecule 1020 by the engineered cells 1000 locates in the cell housing 11. A sensor 21 for sensing the reporter agent/molecule 1020 also locates in the cell housing 11. Both the stimulator and the sensor locate in vicinity to the engineered cells 1000 such that they effectively stimulate the production of the therapeutic agents 1010 and the reporter agent/molecule 1020 and sensing the production of the reporter agent/molecule 1020. At least one side of the implantable device 10 are coated or encapsulated with a permeable material/membrane 23 which shield the implantable device 10 from the immune system/cells of the user.

FIG. 3B provides an illustration diagram of another embodiment of the implantable device 30 which has multiple cell wells/housings 31. The implantable device has more than one cell well/housing 31 attached to an electronic layers 40. Each of the cell well/housing 31 has an individual stimulator 37 and an individual sensor 41. In one embodiment, the stimulators 37 and sensor 41 locate in vicinity to the engineered cells 1000 such that they effectively stimulate the production of the therapeutic agents 1010 and the reporter agent/molecule 1020 and sensing the production of the reporter agent/molecule 1020. In one embodiment, cell wells/housings 31 contain engineered cells producing same therapeutic agent 1010. In another embodiment, different types of engineered cells 1000 producing different therapeutic agents 1010 may be each contained in a separate cell well/housing 31, such that the each type of engineered cells may be individually controlled by the stimulator and the sensor in each cell well/housing 31, so as to produce a particular therapeutic agents 1010 for a specific amount and/or at a specific time different from that of in the other cells/housings.

FIG. 3C depicts an alternative embodiment showing a subcutaneous NTRAIN device 110, including a cross-section that depicts method of operation and associated tasks for engineered components in accordance with an illustrative embodiment. The implanted subcutaneous device includes (i) genetically engineered allogeneic mammalian cells programmed to deliver peptide therapeutics in accordance with an optical trigger 117, (ii) hybrid synthetic biology/bioelectronic feedback control to provide precision dosing 121, (iii) O₂ generation capabilities/device 115, (iv) immune-isolating materials for enhanced cell viability and protection 123, (v) a custom application-specific integrated circuit (ASIC) 125 for low-power feedback control, temperature sensing, and power management, (vi) mm-scale magnetoelectric transducers 126 for wireless power and data/controls downlink, and (vii) a near field communication (NFC) coil 127 for wireless data uplink. In alternative implementations, the device can have fewer, additional, and/or different features.

In one embodiment, the implanted device is approximately 0.8 cm×3 cm, with a thickness of about 2-3 mm, and bendable over a 1 centimeter radius of curvature. In alternative embodiments, different dimensions and/or radius of curvature may be used. Each cell well/housing 111 can include one or more isolated compartments (or enclosures). In one embodiment, each cell well/housing houses about 240 k cells, 2×2×1 mm in size. Alternatively, a different number of cells and/or a different compartment size may be used. At the base of the compartment is a bioelectronic carrier, on which control LEDs (stimulator) 117 initiate and stop peptide production. Specifically, an LED/photodiode pair (sensor) 121 is used to probe production of destabilized fluorescent proteins which are produced as a proxy for the delivered peptide, providing optical feedback of production levels, for closed loop dosage control. The compartment also contains O₂ generating particles or an O₂ generating electrochemical device 115 in one embodiment, which allows the system to have increased density of engineered cells within the chassis. In an illustrative embodiment, the housings that form the cell compartments can be made opaque by using opaque PDMS walls 113 between the compartments 111 to minimize crosstalk of the optical control signals between cell compartments.

In one embodiment, the implantable device can be implanted subcutaneously, pericardially, intracranially, or intraperitoneally for delivery of the therapeutic agents, so as to customized to the subject's needs. In one embodiment, the implantable device can be implanted in a proper location for delivering the therapeutic agents either locally or systematically.

FIG. 3D illustrates another embodiment of the invention. Instead of an implantable device, this embodiment is directed to a wearable external device housing and controlling the engineered cells for production of therapeutic agents. The external wearable device 50 may include (i) a cell housing/cartridge 51 containing engineered cells, (ii) a replaceable cell media cartridge 52, (iii) a pump 53 pumping the media from the media cartridge 52 to the cell housing/cartridge 51, (iv) a cannula 58 extending from the cell housing/cartridge 51 and providing the therapeutic agents to a user, (v) a filter between the cell housing/cartridge 51 and the outlet of the cannula 58 for removing unwanted agents and compositions, (vi) a stimulating system 57 providing light source to the engineered cells housed in the cell housing/cartridge 51, (vii) a sensing system 61 detecting the fluorescent signals produced by the engineered cells for feedback control, (viii) a control unit 63 controlling the stimulation system 57 and sensing system 61, and (ix) a battery unit 65 providing power supply.

In this embodiment, the engineered cells are housed and supported in the wearable external device 50, which delivers the in situ synthesized and excreted therapeutic agents in a regulated manner via the cannula 58 into the body, e.g., subcutaneously, intraperitoneally, and etc.

In one embodiment, the cell housing/cartridge 51 can be a separate, replaceable modular chamber, so as to flexibly customize the production of the therapeutic agents. In one embodiment, the cell housing/cartridge 51 includes microcarriers.

In one embodiment, the stimulating system 57 providing light source(s) of one or more wavelength is disposed in vicinity to the cell housing/cartridge 51. The stimulating system 57 and the cell housing/cartridge 51 are aligned in a manner maximizing the engineered cells' exposure to the light source, e.g., substantially parallel with each other.

In one embodiment, fresh media can be exchanged in the cell housing/cartridge 51, and an on board pump 54 circulated fresh media through the cell housing/cartridge 51 and carries the excreted therapeutic agents through the filter 56 and into the body through the cannula 58. In one embodiment, the replaceable cell media cartridge 52 is replaceable and detachable modular, and the media inside the cell media cartridge 52 can be refilled or replaced.

In another embodiment, a user's interstitial fluid can be collected into the device through a different cannula, circulated through the cell housing/cartridge 51 and then through the filter, before being transferred back into the user's body through the cannula 58 or a separate delivery cannula attached to the cell housing/cartridge 51.

The pump system 54 and the control unit 63 are housed in the wearable external device 50. The battery 65 provides power supply to the wearable external device 50. The battery is replaceable and/or rechargeable.

The wearable external device can be worn by a user or can be attached to the user's skin with adhesive.

The engineered cells housed in the wearable external device 50 have optogenetic system which controls the production of one or more therapeutic agents upon receiving the light signal from the stimulating system 57. The coordination between the optogenetic system in the engineered cells and the stimulating system 57 and sensing system 61 is the same as described for the implantable device in FIGS. 3A-3C.

In this embodiment, the wearable external device relieves the demand for the immune-isolating barrier and the external hub, which may be necessary for certain embodiments of the implantable device. In addition, the wearable external device permits a more flexible size and design choice for the cell housing/cartridge 51.

### Implementation and Operation: Implantation, Implementation, and Life Cycle

In an illustrative embodiment, implementation of the proposed system involves implantation of the subcutaneous device in a subject. The subcutaneous device can be implanted via an outpatient procedure at approximately 2 cm or less below the skin in the abdomen. This implantation location can vary, and depends on the balance of comfort/adoption and systemic delivery efficacy. Additionally, in alternative embodiments, a different implantation location may be used such as omentum, fat, muscle, brain, heart, skin, hips, joints, etc. During use, the implanted device can be secured in a subcutaneous pocket to prevent movement. In some embodiments, the user is outfitted with an external hub in a harness and provided startup operation instructions via an application running on a user device in one embodiment. These instructions guide the user in how to place the external hub by monitoring a power coupling between the implant and the external hub.

In some embodiments of the device the current state of the patient would be evaluated before therapy in order to improve therapeutic timing and dosing. Generally, parameters for determining the dose, timing and etc. of a therapeutic agent delivery schedule are either detected by the external hub via sensing the relevant parameters, e.g., heart rate, blood pressure, core temperature activity status, locations of the user, and etc., or entered manually by the user or another via the external hub. Based on these parameters, a customized therapeutic agent delivery schedule is determined by the control unit of the implantable device or the external hub. Therefore, the delivery schedule can be precisely customized to the user's situation. Once the delivery schedule is determined, the user would be asked to initiate the schedule in the app or directly in the external hub, and confirm its execution by engaging a button on the external hub. The therapeutic schedule is then stored on board the external hub, which triggers the therapy at the appropriate times. Cancellation can be done by the user at any time through external hub or the app.

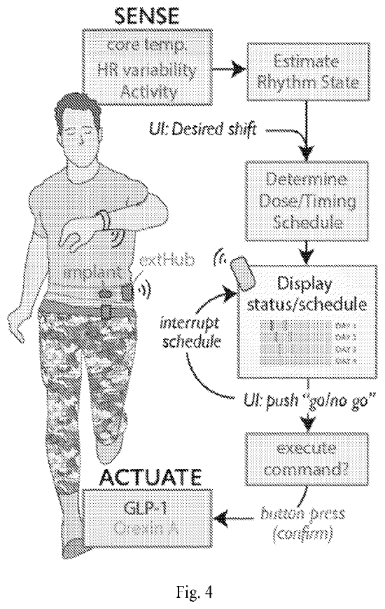

In one illustrative example regarding circadian rhythm control, before first therapeutic activation of a system designed to control circadian rhythm, the user undergoes a baseline period of approximately 3 days (typically at least 1 day) to establish circadian phase with respect to light/dark cycle. When requesting therapy, the user enters a value for a magnitude of an upcoming or recently experienced clock shift in terms of number of time zones, numbers of hours, etc. The magnitude value can be entered in an application in communication with the external hub and/or subcutaneous device, or it can be entered directly into the external hub. The ideal therapeutic schedule (dose, duration, and timing of both peptide therapeutics) is determined by the device based on the magnitude of the clock shift. The user is asked to initiate the schedule in the app or directly in the external hub, and confirm its execution by engaging a button on the external hub. The therapeutic schedule is then stored on board the external hub, which triggers the therapy at the appropriate times. This procedure allows the therapy to be scheduled for delivery at times that may be inconvenient for the user to initiate. In an illustrative embodiment, the user is able to cancel a set schedule at any time using either the external hub controls or the application. Measurements from sensors confirm the progress of entrainment. Additional instructions related to suggested behavior can be implemented at the application or external hub level as appropriate (e.g., suggested use of sunglasses). At any time, the user can input a new shift, for example, return home, and initiate a new therapeutic schedule. FIG. 4 depicts operations performed to implement the proposed hybrid bioelectronics system in accordance with an illustrative embodiment. It is to be understood that other procedures, schedules, and user interaction can be used to treat other conditions.

In an ideal use case, the subcutaneous device can be implanted for a needed duration of time (e.g., length of a deployment, length of a project or job, etc.) and explanted via outpatient procedure once the duration of time ends. Depending on the materials used and the implementation, the proposed system can have a 60 day lifetime, a 130 day lifetime, a lifetime measured in years, etc. For example, it is anticipated that, using the technology described herein, the system could last for years and that repeated administration would be possible. In one embodiment, the engineered cells can be developed to include a genetically inducible safety kill switch to ensure that the cell therapy can be terminated should there be an untoward event during patient use. In the event that the device needs to be rendered non-functional, kill switch activation is initiated by an FDA-approved small molecule biologic. In an illustrative embodiment, viability of cells can be tracked optically to confirm efficacy of the kill switch.

### Engineering Cell-Based Drug Factories

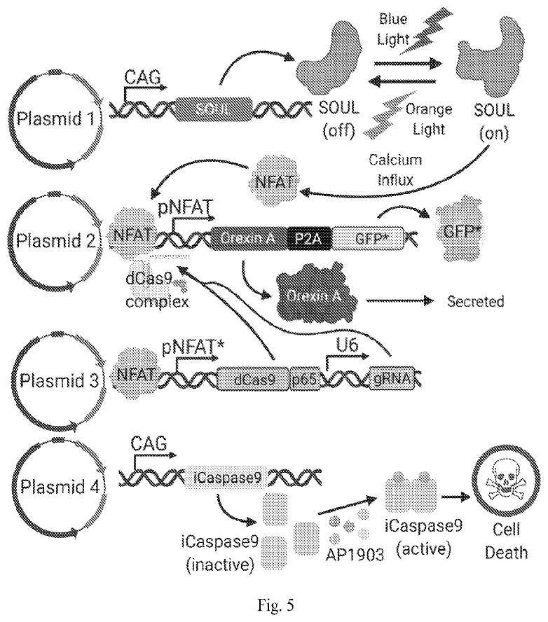

In an illustrative embodiment of a system used for circadian rhythm control, ARPE-19 cells are engineered to produce high levels of the desired therapeutic proteins (e.g., GLP-1 and Orexin A) on an optical trigger. In one embodiment, a melanopsin based optogenetic system can be used. In other embodiments a step-function opsin or dimerizable transcription factor (e.g., EL222) or split transcription factor (e.g., PhyB-TAD, DBD-PIF6) can be used. Additionally, the cells can be engineered to co-express a fluorescent reporter protein, for example, a destabilized GFP (GFP*) in a fixed ratio with GLP-1 and Orexin A, such as 1:1, allowing the system to observe the expression of GLP-1 and Orexin A in real time by using the easily readable destabilized GFP* fluorescence as a proxy. Additionally, a small-molecule-inducible kill switch can be engineered into the cells to allow for easy termination of the cells, rendering the device inactive. FIG. 5 is a graphical depiction of the proposed synthetic biology circuit for optogenetic control of the peptide therapeutic Orexin A in accordance with an illustrative embodiment. Preliminary data demonstrates the utility and feasibility of this architecture.

### Optical Induction of Production

Each of the engineered cells have an optogenetic system. Using engineered cells enables the use of an optogenetic control system to control and produce the desired therapies. Using optogenetic systems, dosing can be controlled by modulating the amount of time that the cells are in the on state. Cells are activated to the “ON” state by exposure to light from LEDs of the stimulating system housed within the bioelectric device. Cells in this “ON” state actively transcribe the therapeutic agents needed to produce the therapeutic.

### Achieving Enhanced Dosing for Therapy

In order to engineer the cells to reach therapeutic dosing, and produce higher quantities of GLP-1 and Orexin A, a catalytically dead version of a CRISPR/Cas9 system (termed dCas9) can be used. The dCas9 system binds to a DNA site-specifically, but does not make any cuts or double-strand breaks. In an illustrative embodiment, the dCas9 can be deployed to recruit transcription activation domains to inserted copies of the NFAT promoter. This will allow amplification of the therapeutic protein and GFP* in a stoichiometrically equal manner amenable to high throughput screening of activation levels and quantification of kinetics. Using this system enables target-agnostic gene activation in a highly specific manner and provides a toolbox of validated synthetic biology tools to tailor activation and kinetics to ideal levels, such as synthetic promoters (NFAT or others), protein degradation tags, and 3′UTR variants among others to facilitate gene amplification only when desired.

### Fluorescent Reporter

Since some embodiments of system utilizes the destabilized GFP (GFP*), co-expressed with the therapeutic, one can observe the production of the therapeutic in real time by observing the fluorescence from the GFP*. Since this protein has a half-life of approximately 7 minutes, it provides an accurate real-time indicator of production levels. This real-time observation of protein production enables feedback for precision dosing control, which can be quantified.

### Resilience to Apoptosis

In an illustrative embodiment, the engineered cells are designed to be durable to apoptosis and senescence, which is important for prolonged and durable expression over the course of usage. To do this, parallel genetic screening is conducted to find genetic modifications that confer resistance to apoptosis and senescence, but that retain the ability for robust kill switch operation. By applying a selective pressure that elicits these phenotypes in the engineered cells (e.g., ARPE-19 cells) and then sequencing them, the system will enrich for cells harboring genotypes robust to these conditions. These genotypes are then recapitulated in an engineered cell line to be encapsulated as a living drug factory.

### Kill Switch

An important consideration with cell-based therapeutics is that the body may reject the cells, leading to a harmful immune response. Additionally, the user may want to render the system inactive. To address this issue, a kill switch is engineered into the cells. Because it has been used in multiple clinical trials and has shown to be safe, the small molecule inducible kill switch iCaspase 9 (iCasp9) can be used in one embodiment. This will allow for the controlled apoptosis of the implanted cells by administering the small molecule AP1903. The molecule can be administered orally or intravenously in some embodiments. Alternatively, in in one embodiment, the system can feature a small on-board payload of the molecule to be released electronically. In other alternative embodiments, a different type of kill switch may be used.

As shown in FIG. 5, in an illustrative embodiment, plasmids are designed for therapeutic protein expression. In one embodiment, 4 plasmids can be used as follows: plasmid (1), codes for a optogenetic system driven by a CAG promoter, to enable constitutive expression of the optogentic system, e.g., production of opsin SOUL; plasmid (2) codes for therapeutic protein (i.e., GLP-1 or Orexin A) linked with GFP* via a linker such as P2A, all driven by pNFAT (activated by NFAT), plasmid (3) codes for dCas9 modification of protein expression levels and can include a unique pNFAT driving transcription of a dCas9 coding region fused to copies of the transcription activation domains p65 or HSF1, or to the human p300 acetyltransferase (p300), plasmid (4) codes for iCaspase 9 being driven by a CAG promoter.

The components of plasmid (3) can be non-virally-derived domains found in human proteins that activate gene expression and will be modulated in copy number to elicit desired amounts of expression. Downstream of plasmid (4) is a synthetic 3′UTR and a U6 promoter driving transcription of the gRNA to target the therapeutic gene promoter for activation. Each plasmid can have a different selection marker (e.g., puromycin, neomycin, blasticidin, and zeocin) and be engineered to have the backbone to allow for lipofectamine transfection with PiggyBac transposase genomic integration.

For cell engineering, in one embodiment, an allogenic human cell line, ARPE-19 (retinal pigment epithelium, or RPE), was chosen because it is non-tumorigenic, displays contact inhibited growth characteristics, is amenable to genetic modification, and has been shown safe in previous human trials. Genetic components can be introduced using the standard piggyBac transposase system to the engineered cells. Other transfection method commonly known in the art can also be used.

In vitro validation and optimization is also performed via fluorescence output and kinetics. For example, the system can measure GFP* after stimulation by blue light and orange light via a live-cell plate reader over the duration of expression. Using this as the basis for further engineering, expression is tuned to be stronger by modifying the dCas9 system as follows: 1) adding more copies of transactivation domains; 2) using stronger activators (e.g., p300); 3) adding more NFAT binding sites to the promoter region; 4) and/or tuning the Kozak sequence. In one embodiment, synthetic 3′UTR variants and degradation tags are used to control stability of the mRNA transcript and protein, respectively.

In vitro validation and optimization of therapeutic outputs is also performed. Therapeutic outputs can be monitored via qPCR, RNA-seq, ELISA, and Western blot across fixed intervals following stimulation by varying durations of blue light and orange light. GFP* production can also be determined via fluorescence reading and compared to GLP-1 and Orexin A production by way of ELISA measurements to confirm a 1:1 stoichiometric ratio. Small molecule kill switch validation can also be performed. To show that the kill switch functions as expected, cells can be cultured with AP1903 (the trigger molecule), and cell viability can be assayed via live-dead staining at various time points after culturing. To determine apoptotic and senescence resistance, the system can also screen for senescence and apoptosis resistant cells using CRISPR guide RNA (gRNA) knockout libraries in combination with doxorubicin, cisplatin, and/or DMSO challenge for a total of 4 different screens (using DMSO as a control). Cells harboring resistance genotyped and iCaspase9 are administered to ensure that the kill switch retains function. Cell fitness, proliferation, viability, and expression levels can be validated through morphological evaluation, BrdU incorporation, MTT assay, and ELISA, respectively.

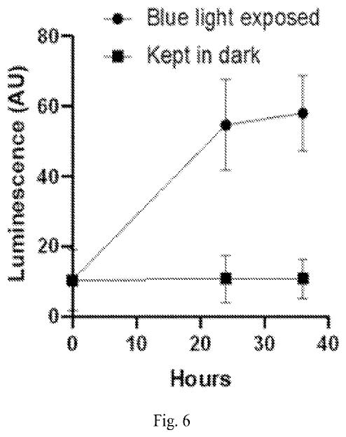

In alternative embodiments, an optogenetic system other than the above-discussed systems to perform cell activation may be used. Other optogenetic system that can be used include melanopsin, EL222 and PhyB/PIF6, which, while they do not have the trigger benefit, but are more established and are shown to work in multiple situations. FIG. 6 depicts preliminary data showing that ARPE-19 cells can be made to express luciferase with high on/off ratio in response to blue light using an EL222 optogenetic system in accordance with an illustrative embodiment.

Precision Control of Dosing based on Optical Feedback

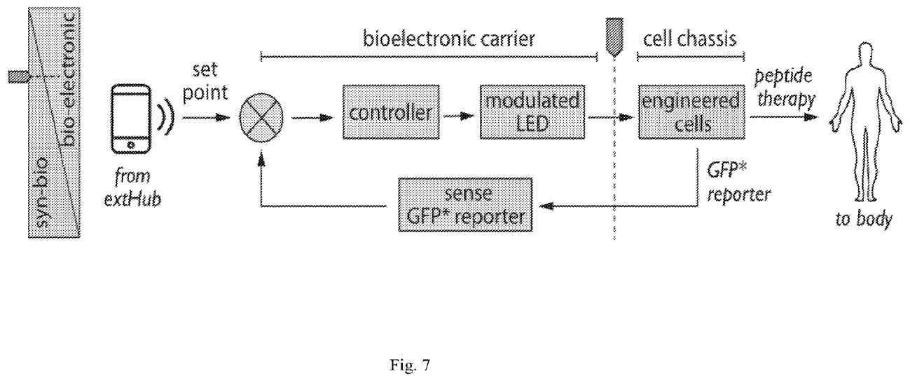

To enable precise and controllable drug production levels despite changes in cell health, stress, and metabolism, a hybrid bioelectronic feedback control system can be created and used. This control system exploits synthetic biology to produce bioactive peptide therapies, and a bioelectronic layer for precise feedback control of production levels. FIG. 7 shows a biohybrid precision control scheme based on co-production of therapeutic peptide and proxy reporter fluorophore (for example, GFP*) in accordance with an illustrative embodiment. As shown, optoelectronics such as photodiode are used to sense and adjust optical stimulation periods to maintain a given setpoint for delivery of therapeutic agents.

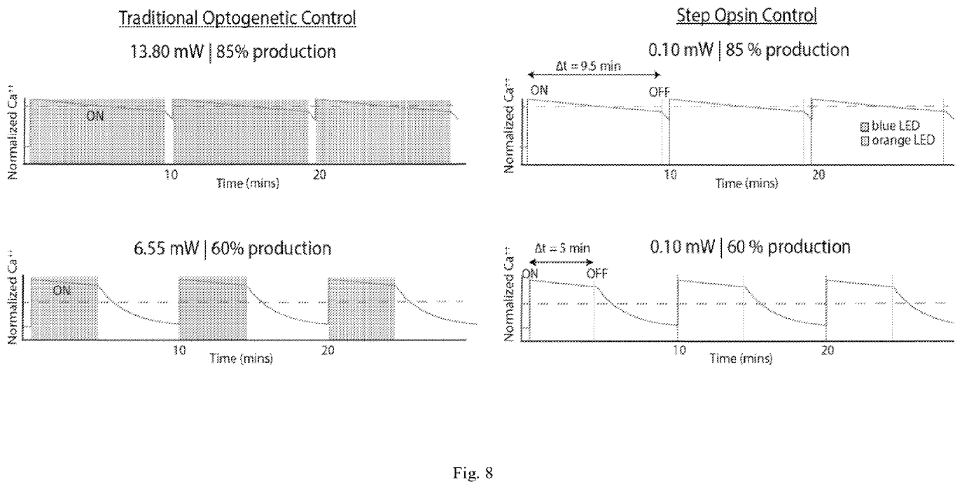

To implement this hybrid feedback control system, light source of stimulation system (LEDs) can be integrated into the implantable device to drive optogenetic channels which regulate therapeutic agents production in the engineered cells. To provide this control signal with minimal power consumption, step-function opsins that are activated and inactivated by different color LEDs are used. Specifically, below each cell housing/well in the implantable device are bonded Individual Cree UltraThin blue LED and Rohm semiconductor PicoLED series orange LEDs. In alternative embodiments, different types and/or wavelengths of light sources may be used. The blue LEDs provide the optical “ON” signal (e.g., 2 s pulse) that turns on the step-function opsin, e.g., SOUL, leading to the elevated calcium levels in the engineered cells, as illustrates in plasmid (1) of FIG. 5, which in turn lead to the production of the therapeutic agents by the engineered cell, as illustrated in plasmid (2) of FIG. 2. The orange LED will provide an optical “OFF” signal (e.g., 2 s pulse) that closes the step-function opsin. By tuning the interval between the ON and OFF signal (Δt), one can control the intracellular Ca++ levels and thus the therapeutic dose with a power savings of approximately 50× compared to traditional optogenetic control, as shown in FIG. 8. To make the dose levels precise, optoelectronics are integrated in the carrier and used to track the fluorescent reporters associated with each therapy. In an illustrative embodiment, the same blue LED used for the ON signal can be used as the excitation light source to track GFP* fluorescence.

FIG. 8 shows a comparison of traditional optogenetic control strategies that use constant illumination to activate the ion channels for the proposed step-function opsin control strategy that utilizes a blue LED to open the light-gated channels and an orange LED to close the channels in accordance with an illustrative embodiment. As discussed, by varying the interval Δt between the ON and OFF, one can control the intracellular Ca++ levels, which in turn determine production levels. As a result, the proposed techniques significantly reduce the average power consumption from >5 mW to 0.1 mW.

In another illustrative embodiment, fluorescence measurements can be made by integrating a green emission light collected by the photodiode over the blue light stimulation block. The LED and photodiode performance can be measured in vitro by comparing fluorometry data to ground truth microscopy data that will measure LED timing, intensity, and fluorescence. In one implementation, lifetime testing can include soaking the encapsulated LEDs in phosphate buffered saline at 37ºC for two months. For in vivo experiments to test photometry, fluorescent microspheres are encapsulated in the chassis and the fluorescence levels from the carrier implanted subcutaneously can be measured.

The GFP* emission is not expected to interfere with the optogenetic system activation state since the emission light is approximately 10⁶ times weaker than the LEDs. Additionally, the feedback controller will account for any non-idealities by adjusting Δt to maintain a desired production setpoint. In an alternative embodiment, an alternative destabilized fluorescent protein such as DsRed-Express that can be excited using the orange “OFF” LED is used, such that any issues regarding cross-talk between the fluorophore and the control signals can be solved.

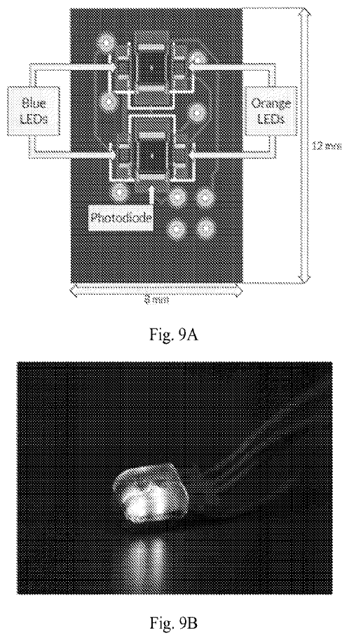

FIGS. 9A-9B show an alternative embodiment of the optical system for the implantable device. FIG. 9A shows the design of the top side of a circuit board with the LEDs and photodiodes. In this embodiment, the light source of the stimulating system and the photodiode of the sensing system are integrated. FIG. 9B shows the LEDs integrating the blue and orange light into one wired prototype LED device with the wires extending out the right side of the LED device.

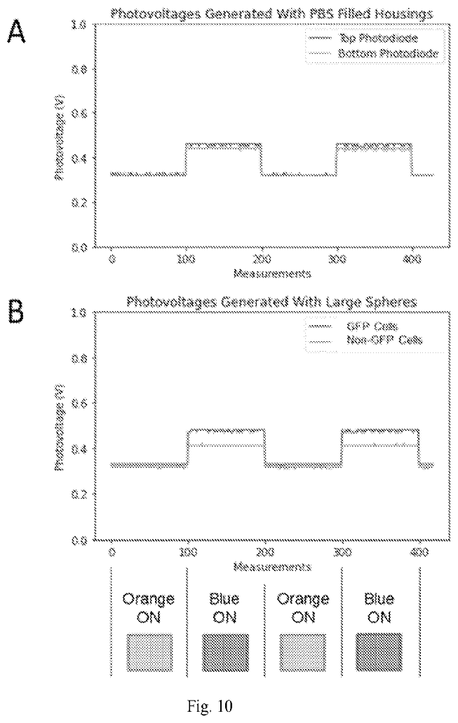

In FIG. 10, the LEDs device as shown in FIG. 9 were used in hybrid bioelectronic device having one or more cell wells/housings 31, as shown in FIG. 3A-D. One cell well/housing 31 contains an alginate capsule filled with non-GFP producing ARPE cells, while the other cell wells/housings contains an alginate capsule filled with GFP producing cells. In this embodiment of the device, the LEDs color was alternated between blue and orange during the “ON” period. As shown in FIG. 10 below, this alternating color between blue and orange showed that when the blue LEDs were illuminated, there was a clear difference in the photodiode readings that was not seen when the orange LEDs were on, as seen in panel B of FIG. 10. This difference is expected to be from the green fluorescent light that is present in the cell well/housing containing GFP producing cell. To further support this claim, a control test was performed by filling the housings with PBS only. As seen in panel A of FIG. 10 reflecting the control test, there was nearly no difference between the photodiode readings of the two cell housings even when the blue LEDs were on. This illustrates the notion that the difference in the readings was caused by the green fluorescence produced by the engineered cells.

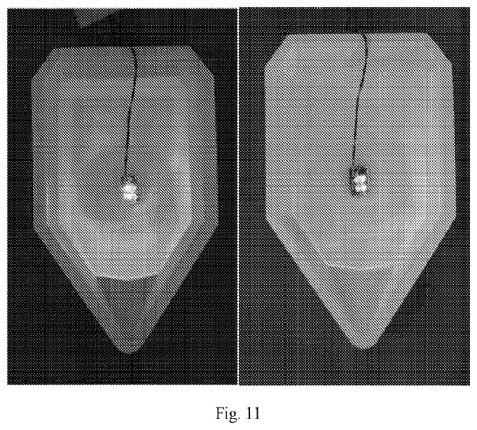

FIG. 11 shows the testing of the integrity of the encapsulation of LEDs device and to ensure that the LEDs and photodiodes would be able to withstand conditions similar to the body. Specifically, a carrier containing the electrical components was placed into a saline solution for 14 days during which time it was continuously powered. If any of the saline solution were to reach the circuitry, the board would short and the LEDs would turn off. The board was visually inspected daily to make sure that the LEDs were still illuminated.

The photodiode performance cannot be directly inspected by the visual inspection of LED light. However, if the saline solution penetrating the carrier, the photodiodes would be shorted due to the saline solution, and thus the LEDs would have turned off. Since the LEDs stayed lit throughout the experiment, it was assumed that the photodiodes did not fail during testing.

The results of the test reflects that the optical source of the stimulating system is capable for proving long term use after being implanted into the body cavity of a user, and the board showed no decrease in performance during or after the 14-day test, as the picture in FIG. 11 was taken after the 14-days period. No images of the board at the beginning of the test are provided since they look the exact same as the images in FIG. 11. This test not only proved that the encapsulation method was successful, but that the LEDs and photodiodes were able to work after encapsulation and even when submerged into a saline solution.

In one embodiment of the invention, similarly sized photodiodes and LEDs were selected for the device so that the LED light would not be blocked by the photodiode. Additionally, the photodiodes are in series with a 100 kΩ resistor to create a measurable voltage. It should be noted that placing the LEDs directly next to the photodiode did lead to noise in the fluorescent photovoltage readings. When the blue LEDs were on, the photovoltage readings averaged approximately 960 mV. The orange LEDs generated a small photovoltage, but since these LEDs will not be illuminated during fluorescent readings, the photovoltage is inconsequential.

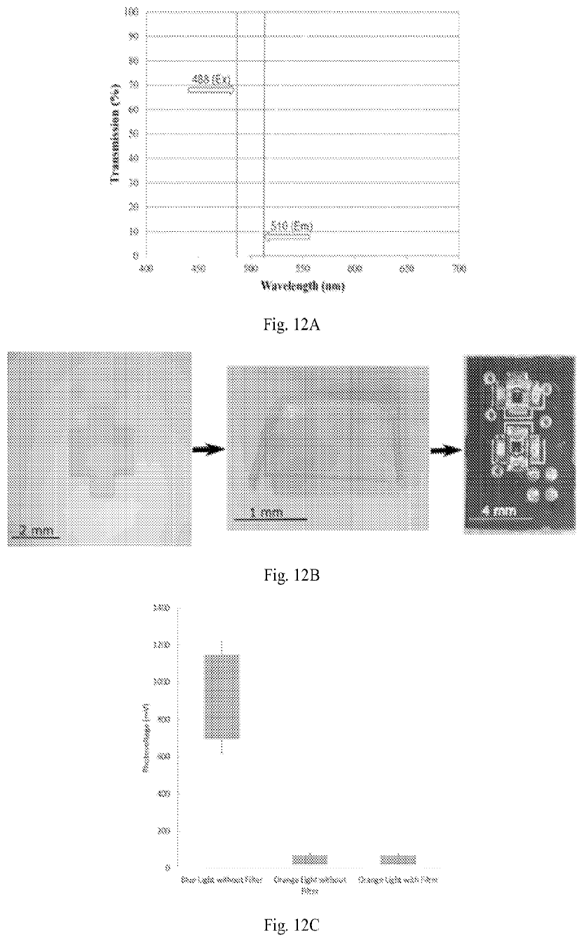

In order to prevent the blue light from reaching the photodiode, the LED light is filtered. The chosen filter was the Wratten 12 filter by Kodak. The Wratten 12 filter is a thin photography filter that acts as a long pass filter with a cut-on wavelength of approximately 500 nm. According to the absorption spectra, the filter should block 99.8% of the blue excitation light. As shown in FIG. 12A, based on the measured absorption curve transmission of the GFP emission light was expected to be 20%. In other embodiment, other filters having approximately same cut-on wavelength may be used.

In one embodiment, the thin film filter was bent into a box shape using a 3D printed mold. The filter was first laser cut into a cross shape and then the flaps were bent with the mold to form the box. This box was then laid on top of the photodiode and adhered to the photodiode using optical adhesive (NOA 84). This procedure is shown in FIG. 12B. In other embodiment, the filter can be formed into other shapes, and cutting and adhering of the filter can be achieved with any known tools in the field of arts.

Photovoltage measurements were taken again after the addition of the filter and the results shows that the filter effectively block the blue light. In particular, As shown in FIG. 12C, comparing to the blue light source without being encapsulated in the filter, the blue light encapsulated in the filter no longer generates any photovoltage, while the orange light still generated the same low photovoltage as before.

### Device Mold and Materials

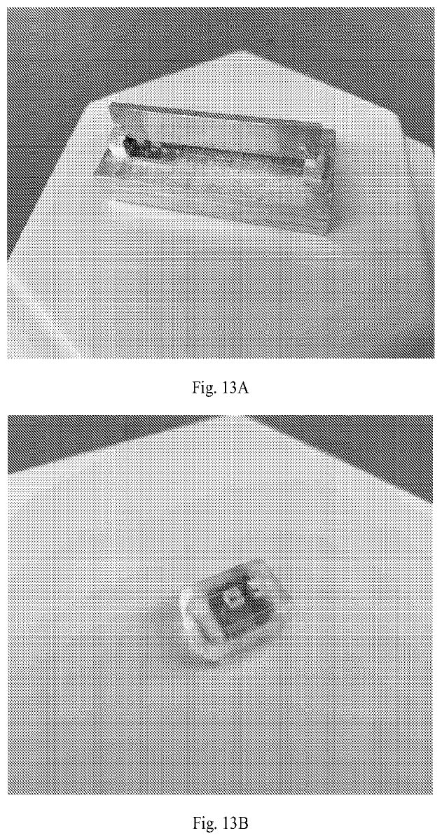

In one embodiment, the structure of the prototype is formed by pouring liquid PDMS into a mold and then letting it cure. Many iterations of mold designs were tested, and once the best design was found, the mold was machined out of aluminum, as shown in FIG. 13A. This was necessary to prevent the carrier from sticking to the mold which was seen with all 3D printed molds.

The shape of the mold allowed for bubbles to become trapped at the top, which would form voids in the device. These voids are points of failure for the device and therefore needed to be removed. A large vacuum glove box was purchased so that the PDMS could be poured into the mold under vacuum. When the mold is returned to atmospheric pressure, many of the bubbles pop and any that do not rise to the surface where they are manually popped with a heat gun. This molding technique has led to bubble-free devices and has expedited the molding process.

The mold design was made so that the circuit board designed in that subtask would fit into the mold and be encapsulated inside of the PDMS. Encapsulation of the circuitry protects the board during use in a saline solution and thus in the user's body. The mold design also took into account the need for cell housings/wells that will be located in the implantable device. The cell wells/housings are formed during molding by placing a comb, similar to electrophoresis gel combs, into the liquid PDMS. The cell wells/housings were successfully formed and are located directly over the photodiodes on the circuit board as shown in FIG. 13B.

Protecting Cells from Immune Response

The implantable device is also designed to protect the engineered cells from the detection and destruction by the host's immune system. Furthermore, when cells are isolated, they may not receive the oxygen and nutrient supply needed to stay alive or productive, which can limit total cell loading, density, and/or efficacy. The present invention balances between protection against the user's immune response and encapsulated cell viability. In one embodiment, the implantable device uses permeable or semi-permeable membranes, such as track-etched polycarbonate or polytetrafluoroethylene (PTFE) have small pores, but at relatively low density, which can limit diffusion of nutrients, oxygen, or produced drug across the membrane, while protect the engineered cells from the user's immune response.

In one embodiment, a multi-layer membrane can be implemented, with hierarchical or layered port structures and/or (bio) material coatings to both isolate the engineered cells from the immune system and promote vascularization near the membrane interface to promote oxygenation. To mitigate the foreign body response (FBR) of the host, the membrane contains sub-micron pores which do not allow for immune cells to transit the membrane, whereas micropores on the tissue side of the membrane will facilitate vascularization. The pore structure can be hierarchically controlled throughout the membrane in a layered manner. In another illustrative embodiment, the membrane surface can be functionalized with either heparin to bind endogenous growth factors or antifouling molecules. Increasing vascularization at the interface facilitates oxygen and nutrient diffusion, hence boosting cell metabolism.

Pore size is characterized by SEM in one embodiment. Additionally, heparin can be further functionalized on the membrane to bind endogenous growth factors, thus, promoting vascularization. Oxygen diffusivity and glucose permeability of the membrane are tested using an oxygen probe and glucose assays to ensure that the requirements for immune-isolating membranes are met (e.g., oxygen diffusivity of 4×10-3 cm/s and glucose permeability of 150 μg/h). Both in vitro human fibroblast (HDF) and endothelial cell (HUVEC) viability and proliferation are also be evaluated. Biocompatibility/stability of the membrane and the ability of the membrane to be combined with cells and in vivo cell viability can further be investigated by histology in both the normal (C57BL/6J mice) and immunodeficient (NU/J mice) mice model. The proposed advances enable enhancement in cell viability and drug production, well over 30 days in vivo.

In another embodiment, commercially available TheraCyte (ePTFE) membranes is modified for enhanced vascularization with nanoporous polymer layers and heparin coating. In one embodiment, to enhance the protection of the engineered cells from the immune system/cells of the user, immune-evasive small molecule coatings is added to the fabrication process for the membrane. It has been shown that a handful of molecules can convey immune-evasive properties to material once coated on the surface. In one embodiment, these small molecule coatings can be added to the material to bolster its ability to mitigate the immune response.

### In Vivo Testing of Drug Delivery

It is important to validate engineered cells in vivo to verify that they display or execute to the proper engineered functions. In one implementation, an experimental group (e.g., mice) has encapsulated engineered cells implanted via an incision/suture procedure, and subsequently have the engineered cells turned on/off using the optogenetic system. Control groups include a first group implanted with encapsulated engineered cells with no optogenetic activation, a second group implanted with just the materials with no cells, and a third group implanted with triazole-thiomorpholine dioxide (TMTD) modified alginate.

When compared to the experimental group, the group implanted with engineered cells with no optogenetic activation allows one to verify the ability of the system to trigger production of the therapeutic protein. Also, when compared to the experimental group and first control group, the second group implanted with just the material allows one to determine whether the optogentic system is “leaking” and producing the therapeutics without being triggered. TMTD modified alginate is a material that is known to not evoke an immune response in mice, and can thus be used as a negative control when looking at the immune response that the material would evoke.

The above-discussed procedure is used to validate the ability of the proposed system to controllably deliver therapeutics, e.g., GLP-1 and Orexin-A, in vivo. Specifically, engineered cells are encapsulated in an immune-protective material and implanted in the subcutaneous space of the test subjects and the skin sutured shut. In vivo light exposure and assaying for protein production is performed. The therapeutics (e.g., GLP-1 and Orexin A in one embodiment) are assayed for via ELISA, while GFP* is assayed via microscopic imaging (e.g., simple fluorescence microscopy, or in vivo imaging system (IVIS)). Implanted engineered cells are exposed to activating light through the skin of the test subject in varying patterns to demonstrate control over expression patterns. Various time points after light exposure are taken to determine the rate that the therapeutics are secreted once the cells are turned on. At each time point, blood samples were taken, along with IVIS fluorescence images. Blood samples are assayed for therapeutics, and the IVIS images are used to quantify GFP* production. ELISA and fluorescence data is compared to calibrate how much fluorescence correlates to a quantity of therapeutic produced. Immune response to implanted encapsulated cells is also measured. Specifically, immune response to the implanted material can be determined by simple microscopy after explant (fibrosis will appear as a layer of biological deposition on the implant if it evoked an immune response). Additionally, immune cell phenotyping can be performed at the implant site to identify any immune cells that are present.

### Wireless Power and Communications and ASIC Development

The present invention also significantly improves the ability to provide efficient, safe, and reliable wireless power, communication, and control for the implantable device through biological tissue. Existing technologies, based on radio frequency (RF) electromagnetics, magnetic induction, or ultrasound, have severe limitations in providing the anticipated 1 mW average power to the implantable device if it is implanted approximatively 1 cm beneath the skin. Electromagnetics (EM) power transfer faces a fundamental tradeoff between antenna size and maximum allowable energy, because tissue absorbs higher EM energy at smaller wavelengths, which are required for small antennas. Ultrasound provides an alternative for wireless power. However, ultrasound suffers from impedance mismatches between air and tissue, and thus ultrasonic gel is typically required, which would be cumbersome during extended use by a system user.

To overcome this challenge, it has been shown that magnetoelectric (ME) materials enable wireless power delivery more than 8 centimeters beneath bone and tissue, which is possible because low-frequency magnetic fields are not absorbed or reflected by the body. It has further been demonstrated that ME materials can be integrated with CMOS chips to create regulated power supplies and transfer data to implants. Importantly, this technology allows one to effectively deliver power and commands to miniature implantable devices without the need for any impedance matching gels or liquids.

Results also suggest that ME transducers are inherently less sensitive to misalignments and angle changes with the external transmitter than inductive coupling, which ensures that the minimum required power can be delivered even under movements of a user. In one embodiment, a near-field communication (NFC) scheme by inductive backscattering can be used for uplink data transmission. The sensitive NFC protocol can be used for the uplink because compared to the power and data downlink, the uplink is less sensitive to loss and alignment errors. The larger antenna and stronger computational and filtering capabilities of the external device allows it to extract the uplink data from noisy backscattered signals.

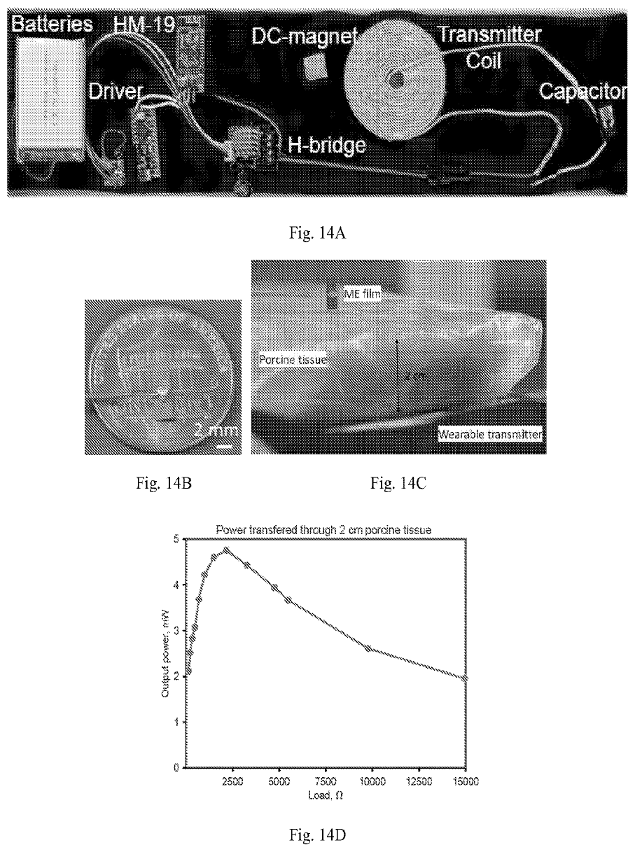

FIG. 14A-D illustrate one embodiment of the ME system. To demonstrate the effectiveness of the magnetoelectric technology (ME) for wireless power transmission through porcine tissue, a ME film was fabricated using a 30 um-thick layer of Metglas 2605SA1 (Metglas Inc) attached using epoxy to a 270 um-thick layer of PZT-5A4E (Piezo Inc.). The sheet is cut using a laser cutter to a miniaturized 7*2.4 mm films, and FIG. 14B shows the size and thickness as compared to a penny coin. For such films, the mechanical resonance frequency is found to be 240 kHz.

The ME films is powered using a low magnetic field that can be generated using a wearable, battery-powered transmitter. To design such a transmitter, rechargeable lithium-ion batteries, H-bridge, microcontroller, Bluetooth module, DC magnet, resonance coil of a circular spiral coil and capacitor bank are integrated as shown in FIG. 14A. To test the proposed system performance, the ME film and the transmitter coil are separated by a 2 cm thick porcine tissue as shown in FIG. 14C. The AC field generated by the coil=1.1 mT whereas the measured open-circuit voltage at the ME film terminal=7 Vp-p.

The output power generated by the ME film depends on the connected load, hence, different resistive loads are connected to the film terminal and the load voltage is measured to compute the output power as shown in FIG. 14D. As can be seen, the output power is greater than 2 mW for the different loading conditions.

One of the main implementation challenges arises from fluctuations of ME induced signals caused by body movements, the stringent power budget, and heating limits of the carrier. One way to mitigate the challenges is to make the system adaptive to external conditions and workloads, which increases robustness and reduces testing/calibration efforts over the conventional design methodology using pre-defined specifications and tolerance. In one embodiment, critical information (system clocks, data decoding thresholds, and transitions between different modes) can be extracted from the real-time ME induced signals. The single-input, multi-output power management unit is adaptive to workload changes and spends minimum power to provide regulated supplies that accurately meet the diverse needs (voltage, ripple, load current) of each module. The goal is to minimize power waste and unnecessary heating due to excessive headroom and quality of outputs.

Moreover, the power management unit is able to monitor and report the received power and produced voltage to enable efficient control of the external hub's transmitter output power, and to provide a measure of the relative distance between the implant and the external hub, which enables simple tracking of the implant. In addition to system-level optimizations, ultra-low-power and highly digital circuit techniques have been developed to achieve comparable performance as conventional high-precision analog circuits at much lower power and complexity. Minimizing the power consumption of all chip components effectively increases the end-to-end efficiency from power source to bioelectronics, leading to reduced heating and better tolerance to body movements, and longer battery lifetime of the external hub.

In certain embodiments, the hybrid bioelectronics system, instead of a wireless power charging system, may have a battery on board. In one embodiment, the battery is replaceable and/or rechargeable.

### External Sensing and Communications (External Hub)

As discussed herein, the external hub is a battery powered wearable that provides power, communication, processing, storage, and a user interface to monitor and control the implant. In some embodiments the external hub is not used. In one embodiment, the external hub clicks securely into a socket in a comfortable, adjustable harness positioned on the abdomen or other area on the host that is proximate to the subcutaneous implant. Alternatively, a different mounting technique may be used. The external hub serves as a gateway for all data and control, and includes a multi modal sensor suite designed to understand human behavior—such as sleep rhythms. The external hub provides controls and a display on the hub itself, in a protected pocket, or by connecting to a phone application. The phone application, beyond providing a mechanism for user input and control, also provides a way for the user to localize the implant and perform initial setup and system configuration. In alternative embodiments, a phone application may not be used, and all control and functionality can be performed through the user interface of the external hub, which can include a touchscreen display, one or more buttons or other controls, etc. The external hub is designed to enable long wear time, provide bio-sensing ability, enable intuitive controls, and be reconfigurable for diverse applications, including supporting the proposed entrainment therapies and future therapies.

In one embodiment, the external hub can be built iteratively, verifying and testing novel functionality as more advanced prototypes are designed. As one example, a desktop, wall powered system with essential components can be designed, so that low level software development can begin at speed. A portable prototype can be developed for testing and validating sensors to monitor circadian rhythms, where the portable prototype includes only the external sensors, and a data acquisition unit/microcontroller with built-in telemetry functions for data offload to a nearby desktop, thereby facilitating circadian rhythm sensing for NHPs. Concurrently, power circuitry and battery lifetime management circuitry can be handled in a more portable prototype holding all functionality beyond just sensing. This also enables harness and enclosure design to begin, and for the external hub to be further miniaturized until the final device with all functionality and user controls is delivered. This sequence of hardware designs enable the handling of various device challenges, which are described below.

In commercial off the shelf wearables, battery lifetime can vary dramatically based on the actions of the user, the software running, the signals environment, and the internal components that are activated. While running out of battery is merely an inconvenience for the average person, for a user of the proposed system it may severely hamper mission/work readiness. Reliability and predictability of battery lifetime for complex cyber-physical systems like the external hub is critical but challenging because of the intersection of software, hardware, and user non-determinism. Therefore, a robust energy model is embedded in the firmware of the external hub that is circadian rhythm and environment aware, enabling prediction of external hub lifetime with high accuracy, such that completion of therapies and the mission/work is ensured. The energy modeling can include complex cyber and physical components, including the implant operation, a physical signals environment, sensing algorithms, user interaction modeling, and therapy delivery. This static model can be augmented by in-situ power measurements and execution traces such that the static model is continuously refined. This energy model is a core portion of the external hub operating system, and can leverage embedded energy models to enable ultra-long (e.g., nine month plus) battery lifetime.

In an illustrative embodiment, the external hub uses non-contact COTS sensors to measure and track diurnal variations in body temperature, heart rate and variability, and physical activity, which are inputs to a model of circadian control that estimates the circadian phase of the wearer. Leveraging the sensing capability of the external hub for circadian phase monitoring, instead of sensing in the implant, allows for a smaller sized, more comfortable, and lower power implant. The system can also use well studied sensors for sleep and activity monitoring, such as ballistocardiography based heart rate sensing and accelerometer based actigraphy. Ballistocardiography (BCG) is a measure of ballistic forces generated by the heart, enabling measurement of R-R interval. Accelerometers can measure these forces even if not directly above the heart, or even attached to the body (for example, R-R interval was captured when an accelerometer was placed under the mattress of a sleeping person). As the system will continuously sense circadian phase, estimates of heart rate will occur even when the wearer is active. As such, a 9-axis inertial measurement unit is used that allows for removal of gravity effects and motion artifacts, and provides orientation of the wearer (from the magnetic sensor), to understand posture. Actigraphy methods can be used to separate sleep activities from confounding activities such as exercise, eating, or walking. Infrared based skin temperature sensors are also included to validate/calibrate internal temperature from the implant, and provide a coarse estimate of heat flux based on known values of thermal conductivity of human skin, which can be used to estimate core body temperature. The external hub captures all raw signals from these sensors, cleans the signals, and extracts relevant biomarkers for sensing circadian phase. Software machine learned models that reduce noise are developed for each sensor.

The user of the proposed system needs mechanisms to control therapies no matter the situation (before a mission, when traveling, while working, in the field, etc.). Physical controls of the system are designed with visual feedback on the external hub to program, stop, and start therapies in the field. In an illustrative embodiment, the controls lie in a protected compartment in the enclosure to ensure no accidental actions. The controls can be mirrored on a smartphone application with the same capabilities in one embodiment. This provides seamless control no matter what situation the user is in. The smartphone application connects with the external hub using an encrypted Bluetooth LE channel in one embodiment, and allows for richer visualization of entrainment progress. As a result, the wearer can understand the effect of designed therapies in real time. This innovative, mirrored, multi-context interaction approach provides a new way to think about and visualize on the go applications for users in high stress, highly mobile environments.

The enclosure for the external hub will protect the circuitry in a slim profile watertight package. Controls can be in a protected pocket to prevent accidental button presses. The enclosure can be designed iteratively, in a large functional form factor, then miniaturized, hardened, and waterproofed. On first use, the external hub can be placed in the harness, and the localization procedure is initiated from the smartphone app (or alternatively on a user interface of the external hub). The application (or external hub itself) guides the user on which direction to move the external hub for optimal power based on measuring received signal strength from the implant NFC uplink. Once the location is set, the harness is tightened to secure the placement. The harness is co-designed along with the external hub based on existing abdomen harnesses that secure items like radio transmitters, etc.

Security of communication, and security of operation are critical for delivery of entrainment therapies. This problem is addressed by the proposed design. Specifically, ME communication is highly localized, making it difficult to emulate control commands sent from the external hub to the implant. A highly sophisticated attacker would need to be uncomfortably close to the implant location to interfere with operation. In an illustrative embodiment, the implant will confirm commands it has received via the NFC uplink with the external hub to ensure the command originated from the external hub. In another embodiment, a co-processor model is used that involves processors operating in tandem to continuously verify software operation. The smartphone application, if used, can connect with the external using an encrypted Bluetooth LE channel, reducing possibility of malicious data exfiltration. Alternatively, a different secure communication channel may be used to perform communication between the user device (e.g., smartphone, tablet, laptop, etc.) upon which the application is located and the external hub. Security checks can also be performed at each layer of the software/hardware stack in the external hub, which will further reduce the possibility of tampering and data exfiltration.

In the event of issues with battery lifetime being shorter than anticipated due to ME power costs, the size and/or shape of the external hub can be adjusted to support a larger battery. Further, if it is determined that the non-contact sensor selection is not sensing heart rate accurately enough for the circadian phase sensing algorithm, electrodes for EKG can be placed on the harness itself, utilizing skin contact.

### Buffer/Nutrient Solution for Cell Wells/Housings

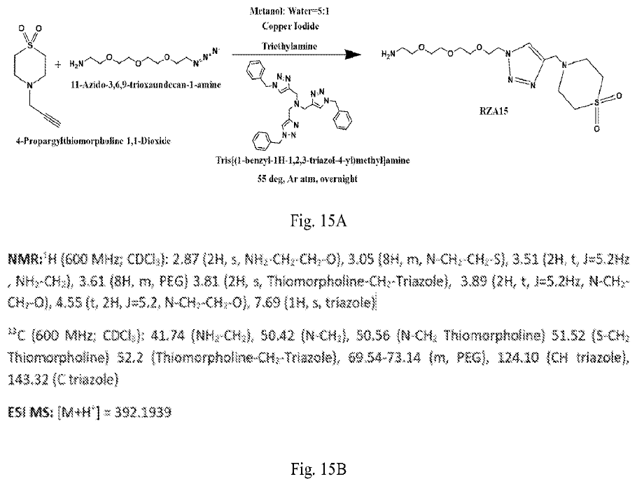

In one embodiment, RZA 15 is used as a suspension solution to suspend engineered cells in the wells/housings. RZA15 is a molecule that will help protect the cells from fibrosis in vivo promoting cell viability. RZA15 was synthesized in the following manner as shown in FIG. 15A.

In particular, in one embodiment for synthesizing the RZA15, 4-Propargylthiomorpholine 1,1-Dioxide (1 eq.) was added to a 250 mL round bottom flask and dissolved in methanol:water mixture (5:1). Consequently, Tris[(1-benzyl-1H-1,2,3-triazol-4-yl)methyl]amine (0.25 eq.), Triethylamine (0.25 eq.), and copper iodide (0.1 eq.) was added. The reaction mixture was purged with argon for 15 mins and cooled to 0° ° C. following which 11-azido-3,6,9-trioxaundecan-1-amine (1 eq., 6.30 g, 28.86 mmol) was added. The reaction mixture was stirred at room temperature for 15 mins and afterward heated to 55° C. for overnight. The reaction was cooled to room temperature and filtered through celite to remove any insoluble part. The filtrate was dried using rotavap under reduced pressure with silica. The crude reaction was then purified by liquid chromatography with dichloromethane: ultra (22% MeOH in DCM with 3% NH₄OH) mixture 0% to 40% on a 120 gm ISCO silica column. The final product was further characterized with ESI mass and NMR mass spectroscopy according to FIG. 15B.

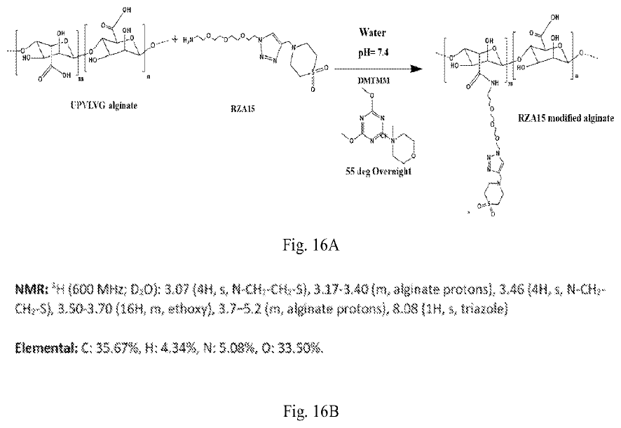

Once the RZA5 is synthesized, it is conjugated to UPVLVG alginate to be used as the hydrogel to suspend cells in the carrier. The conjugation was then carried out in the following manner as shown in FIG. 16A.

In one embodiment for conjugation, in a round bottom flask, 2 g (leq) of UPVLVG (BP-1903-04; Novamatrix) was dissolved in water (75 mL). Then RZA15 small molecule (3.99 g, 10.20 mmol, leq) was dissolved in water by vortexing and the pH was adjusted to 7.4 using HCl. Then RZA15 aqueous solution was slowly added to the UPVLVG while stirring. Subsequently a solution of (4-(4,6-dimethoxy-1,3,5-triazin-2-yl)-4-methyl-morpholinium chloride) (DMTMM, 0.5 eq.) was added dropwise to the mixture of UPVLVG and RZA15. The reaction was heated to 55° C. and stirred overnight. The solution was filtered through a cyano-silica and dialyzed in a 40 cm long 10-12K pretreated dialysis tubing in a beaker using saline (2 days) and mili-Q water (3 days). The dialyzed solution was frozen at −80 degrees and lyophilized until dry. The final product was then characterized by NMR and elemental analysis with elemental analysis showing a 16% modification of the alginate material. NMR and elemental characterization of the conjugates is shown in FIG. 16B.

### Supporting and Controlling Cells in a Bioelectronic Carrier

One key challenge for implementing a feedback control on the bioelectronic carrier is to engineer sufficient energy efficiency to operate within the desired wireless power budget. In one embodiment, a step-function control strategy is used to reduce the power needs. In one embodiment, the step-function control strategy reduces the power needs by approximately 50×. In addition to reducing the average power consumption, the inventors also designed the system to minimize the peak instantaneous power requirements, to avoid the need for large energy storage elements on the device. To reduce the need for large peak power, LED control sequences can be interleaved within 10-minute blocks so that only one LED will be active in any cell housing/well at any one time. Additionally, the seconds scale activation and inactivation pulses required to turn on and off the optogenetic system channels can be converted into high frequency (50 Hz) pulse trains with the same total energy. In one embodiment, these two strategies offer an approximately 10× reduction in peak power that allows for miniaturization of the charge storage elements. Light-leakage and optical cross talk between wells can be reduced in some embodiments by doping the PDMS with gold NPs that will render the walls opaque without compromising the permeability and elastomeric properties of the material. To ensure that the LED and photodiode remain functional inside the body, Parylene C, Parylene N, SiC (Silicon Carbide), and Medical grade epoxies can be used for effective bioencapsulation. In addition, the photodiode can be coated with a combination dielectric and absorption filter which has been shown to be effective for on-chip fluorescent imaging.

To enable the proposed high-density cell loading, oxygen can be supplied to the encapsulated cell well/housing. This can be accomplished via electrolysis of water at adjacent microelectrodes in one embodiment. In another embodiment, O₂ gradients are tailored and maintained to optimize the performance of encapsulated cell well/housing by precisely tuning the platform's electrode size, spacing, and power supply. The use of selective polymer membranes can be used to minimize reactive byproducts and protect the device from bio-fouling. A primary goal is to generate enhanced oxygen concentration on a small device footprint and with a low overall power budget. In alternative embodiments, depending on the type of cell(s) being used or the cell density, O₂ may be supplied with oxygen producing CaO nanoparticles, or O₂ may not be supplied to the cell well/housing at all.

### Cell-Carrier Integration and Validation

Another challenge in creating the proposed system is integration of the bioelectronic cell housing with the living engineered cells. To overcome this challenge, the inventors developed a biohybrid co-fabrication strategy using an established program for non-traditional fabrication of bioelectronics and the developed cell encapsulation strategies. In one embodiment, the carrier and cell chassis are fabricated separately and combined using a biocompatible epoxy-based glue. The system can be sterilized using EtOH and placed in a double sterile sealed container. Immediately prior to implantation, the system can be unsealed and cells can be injected through injection ports in the carrier and sealed with the FDA-approved biocompatible epoxy glue by the clinician at point of application. The system was designed in this manner to account for ease of commercial manufacturing and shipping, and in accordance with regulatory guidance.

### Cells Loading Aid Device

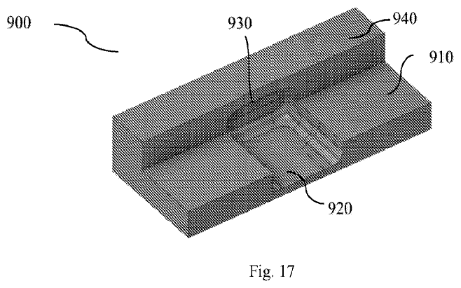

FIG. 17 shows a cell-loading aid device 900 responsible for loading the engineered cells into the cell wells/housings of the hybrid bioelectronic device. The aid device 900 has a base 910 and a guide 940 attached to one longitude side of the base 910. The base 910 has a niche 920 for receiving the hybrid bioelectronic device. In the guide 940, there exists at least one hole 930 through which the engineered cells are injected into the cell wells/housings.

### Computing System

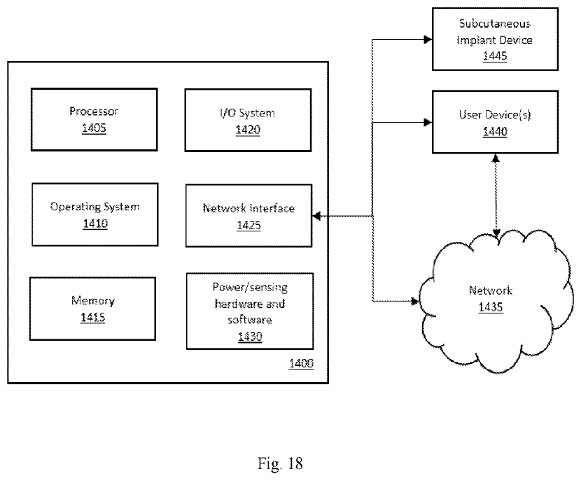

In an illustrative embodiment, components of the proposed system such as the subcutaneous implant device, external hub, and/or associated user device can include and/or be in communication with one or more computing systems that include a memory, processor, user interface, transceiver, and any other computing components. Additionally, any of the operations described herein may be performed by the computing system(s) of these components. The operations can be stored as computer-readable instructions on a computer-readable medium such as the computer memory. Upon execution by the processor, the computer-readable instructions are executed as described herein. As an example, FIG. 18 is a block diagram of a computing system to perform operations described herein in accordance with an illustrative embodiment.

Specifically, FIG. 18 depicts one embodiment of a computing device 1400 (e.g., an external hub) in direct or indirect communication with a network 1435, one or more user devices 1440, and a subcutaneous implant device 1445. The user device(s) 1440 can include a smartphone, tablet, laptop, smartwatch, activity tracker, or other user device that is in communication with the computing device 1400. As discussed, the user device(s) 1440 can include an application that interfaces with and controls the computing device 1400.

In this illustrative embodiment the computing device 1400 includes a processor 1405, an operating system 1410, a memory 1415, an input/output (I/O) system 1420, a network interface 1425, and power/sensing hardware and software 1430. In alternative embodiments, the computing device 1400 may include fewer, additional, and/or different components. The components of the computing device 1400 communicate with one another via one or more buses or any other interconnect system. Although not depicted in FIG. 18, it is to be understood that the subcutaneous implant device 1445 and the user device(s) 1440 can similarly include computing components such as a processor, an operating system, a memory, an input/output (I/O) system, a network interface, power/sensing hardware and software 1430, and/or any of the other computing components described herein.

The processor 1405 of the computing device 1400 can be in electrical communication with and used to control any of the external hub components described herein. The processor 1405 can be any type of computer processor known in the art, and can include a plurality of processors and/or a plurality of processing cores. The processor 1405 can include a controller, a microcontroller, an audio processor, a graphics processing unit, a hardware accelerator, a digital signal processor, etc. Additionally, the processor 1405 may be implemented as a complex instruction set computer processor, a reduced instruction set computer processor, an x86 instruction set computer processor, etc. The processor 1405 is used to run the operating system 1410, which, as discussed herein, can be a custom operating system specific to the requirements of the external hub.

The operating system 1410 is stored in the memory 1415, which is also used to store programs, sensed patient data, algorithms, network and communications data, peripheral component data, and other operating instructions. The memory 1415 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc.

The I/O system 1420, or user interface, is the framework which enables users and peripheral devices to interact with the computing device 1400. The I/O system 1420 can include one or more keys or a keyboard, one or more buttons, one or more displays, a speaker, a microphone, etc. that allow the user to interact with and control the computing device 1400. The I/O system 1420 also includes circuitry and a bus structure to interface with peripheral computing components such as power sources, sensors, etc.

The network interface 1425 includes transceiver circuitry that allows the computing device 1400 to transmit and receive data to/from other devices such as the subcutaneous implant device 1445, the user device(s) 1440, remote computing systems, servers, websites, etc. The network interface 1425 enables communication through the network 1435, which can be one or more communication networks. The network 1435 can include a cable network, a fiber network, a cellular network, a Wi-Fi network, a landline telephone network, a microwave network, a satellite network, etc. The network interface 1425 also includes circuitry to allow device-to-device communication such as near field communication (NFC), Bluetooth® communication, etc.

The power/sensing hardware and software 1430 can include hardware, software, and algorithms (e.g., in the form of computer-readable instructions) which, upon activation or execution by the processor 1405, performs any of the various operations described herein such as sensing data, receiving sensed data, performing analyses of sensed data, generating control signals, generating power and controlling power usage, etc. The power/sensing hardware and software 1430 can utilize the processor 1405 and/or the memory 1415 as discussed above.

In an illustrative embodiment, the subcutaneous implant device 1445 can be any of the implant devices described herein, and can include any of the functionality/components described herein. In one implementation, the subcutaneous implant device 1445 can include an electronic layer that can include one or more actuators to control cell production, one or more sensors, an ASIC, a processor, a memory, a battery, a transceiver, etc. Attached to the electronic layer is a biological cell layer that includes engineered cells. In an illustrative embodiment, the engineered cells are within a hydrogel that forms at least a portion of the biological cell layer. The hydrogel can be within a chamber that is accessible to the sensor(s) and/or actuator (e.g., a transparent bottom of the chamber can be used if the cells are actuated via optoelectronics). In another embodiment, the implant device 1445 can be separated from the body by a membrane that allows diffusion of small molecules but blocks cells (i.e., does not allow immune cells in, or engineered cells out).

### Circadian Rhythm Control

In one exemplary implementation, the proposed system can be used to help control the circadian rhythm of the subject in which the system is implanted. For example, the system can be used to accelerate human adaptation to a new time zone or work schedule by synergistically shifting central and peripheral circadian clocks. While various examples and implementation details are provided herein with respect to control of circadian rhythm, it is to be understood that the proposed system is not limited to circadian rhythm applications. Rather, as discussed herein, the proposed implantable cell generation system can be used to provide pain relief, fight diseases, cure disorders, provide immune response control, treat infertility, etc.

It has been shown that desynchrony between multiple circadian clocks and the light/dark cycle can result in decrements in mental performance and health and act to hinder entrainment. Traditional methods to accelerate adaptation, or entrainment, focus on light therapies or fixed-dose, single-target medications that may not be ideally timed or efficacious. These approaches focus largely on central clock-driven rhythms such as the sleep/wake cycle, without considering adverse effects of misaligned internal clocks that entrain more slowly than central hypothalamic clocks. To most effectively accelerate entrainment, the proposed system engages the entire network of a host's central and peripheral clocks with precise control of the timing and dose of peptide therapies. This level of precision exceeds what is possible with current therapies. Namely, the proposed system is able to achieve 1) specific biological action on select target receptors or molecules that cannot be accomplished with current bioelectronics, and 2) precise control of timing and dosage that cannot be accomplished with current synthetic biology.

### Sensing Circadian Phase

In an illustrative embodiment, a multi-sensor fusion strategy is used to accurately measure the phases of the multiple 24-hour rhythms that are disrupted by long-distance travel and late-night operations. Specifically, biophysical, physiological, and behavioral markers are measured to track multiple rhythms and overcome confounding factors (like physical activity) that could mask a CR measurement based on any singular sensing modality. Real-time assessment of circadian phases of both central and peripheral clocks enables precise timing of therapeutics. Existing, robust sensor technologies, including internal and skin surface temperature, 9-axis inertial measurement units, and heart rate sensing techniques are used. These parameters exhibit robust circadian rhythms and are can be used to access the circadian timing. The timing of these rhythms are regulated by distinct SCN output pathways as well as by different local tissue physiology and environmental timing cues, thus together depicting the status of the hierarchical circadian system as a whole. In alternative embodiments in which the cell factory is used to treat pain, manage diseases, etc., sensor data may not be used. In other embodiments, different types of data may be sensed, specific to the non-circadian application.

The sensor data is used as inputs to a well-established model of circadian control to produce an estimate of circadian phase and predict phase shifts in response to stimuli and delivery of therapies. The most common approach is to model circadian control as a limit cycle oscillator. These models simulate the rate of change in state variables (e.g., core body temperature, activity, heart rate) as functions of the current status of state variables plus “drives” from external stimulus (e.g., entraining agents such as light or therapeutic peptides). Using such models, the current internal timing of the animal (i.e., circadian phase) can be resolved from the values of the state variables, and predictions of phase-shifts induced by light and/or therapeutic peptides can be made at any phase of the cycle.

Since diurnal variations in each of the selected biomarkers reflect different facets of circadian control, a unified measure of circadian phase is generated that incorporates all 3 biomarkers as state variables in a single “limit sphere” model. Each state variable can have a different driving function in response to light or peptides to represent their different rates of entrainment. In this way, the model can also detect misalignment between the phase reference points of different state variables during re-entrainment. Estimates of circadian phase are measured as a percent error relative to reference measures obtained with a fully implanted sensor system that is the standard used in most sleep studies. In addition to data collected for model development and testing, data of peptide-induced phase shifts in mice can also be used to facilitate the selection of model framework. The model can be fine-tuned to direct daily peptide treatment schedule to achieve accelerated entrainment.

Testing can be performed in subjects instrumented with a standard suite of sensors used in sleep research, including electroencephalography (EEG), electrooculography (EOG, eye-movements), and electromyography (EMG, neck muscles) that will provide gold-standard reference measures of circadian phase for comparison with the NTRAIN sensor suite. Data can be collected continuously around the clock via COTS implantable telemetry devices that integrate all of the required sensing functions in a fully implantable, battery-powered package that can transmit data continuously for long periods of time. An initial prototype of the NTRAIN sensor set (external hub) will be implanted to verify sensors in vivo prior to full integration of the external hub. In addition to performing continuous monitoring of physical and physiological biomarkers, established behavioral assays of cognitive function (e.g., working memory, attention) are also implemented to measure changes in cognitive performance throughout the circadian cycle. These tests can be incorporated into the daily enrichment schedule for the test subjects, which minimizes stress and improves psychological well-being. The enrichment schedule includes social interaction, physical activity, sensory stimulation, food, and cognitive/occupational activities. Thus, the cognitive testing protocols provide enrichment in all five categories, and the testing data generates operationally-relevant, performance-based measures for evaluating the effects of CR-entrainment therapies.

Circadian phase-sensing is used for determining the type, timing, and dose of therapies to deliver. Successful completion of this task depends on 3 key factors of low to moderate risk and are discussed in order of decreasing risk. First, obtaining reliable measures of circadian biomarkers is moderately risky, particularly in large animals. An established COTS system (DSI telemetry) that has been used for similar long-term monitoring studies in many species, including non-human primates, can be used. Second, the circadian control model is essential for interpreting the biomarker data. Accurate phase predictions are essential and there is a low risk that the algorithm will not generalize across all conditions. However, this risk is thought to be low to very low.

### Circadian Rhythm Therapeutics Delivery

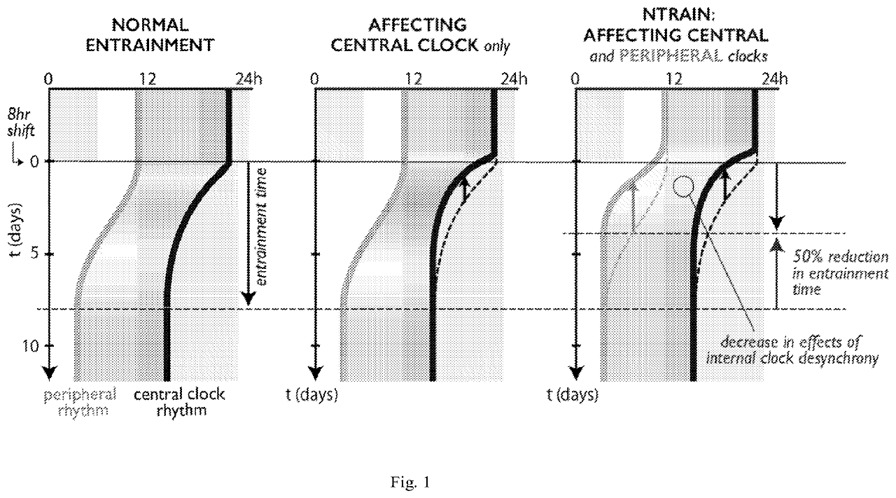

In one exemplary embodiment of multiple therapeutic delivery, multi-clock targeting with precision timing for circadian rhythm regulation can be performed by the proposed system. For example, in the circadian rhythm example, by targeting both the central and peripheral clocks, the hybrid bioelectronics system of the present invention provides synergistic effects towards enhanced entrainment, as shown in FIG. 1. However, because the same therapy applied during different phases of a circadian rhythm can have both phase-advancing or phase-delaying effects, it is important to validate therapeutic efficacy in terms of its administration window. The system uses phase response curves (i.e., the phase shift induced by therapy as a function of the phase of delivery), combined with real time sensing of internal body temperature and/or commercial off the shelf (COTS) wearable sensors to inform actuation-timing for most effective delivery of therapies. FIG. 1 depicts phases of peripheral and central clocks in response to an 8 hr shift, for normal entrainment (left), providing therapy affecting only the central clock (middle), and the approach of the hybrid bioelectronics system of the present invention (right) with therapy targeting both central and peripheral clocks in accordance with an illustrative embodiment. In FIG. 1, the fill color green represents normal phase relationship, and red represents misaligned phases. The system of the present invention rises above the current state-of-the-art in circadian rhythm management because it delivers a personalized therapy with precision dosing and timing for maximum efficacy. This is not possible with single-dose approaches that act only on sleep/wake rhythms.

In the illustrative embodiment for the circadian rhythm application, the therapeutics targeted for production and delivery by the engineered cells are GLP-1 and Orexin A. In alternative embodiments, different types of therapeutics may be produced. Production of such peptides presents an inherent advantage, especially in the application of circadian management. The peptides are produced by mammalian cells and thus are native, non-recombinant peptides. Unlike recombinant variants, GLP-1 and Orexin A have short metabolic half-lives (GLP-1, 4.6-7.1 min; Orexin A, 27 min), making their use for entrainment more effective. Such half-lives are long enough to reach target tissues, short enough to have a precisely timed phase-shifting action, and are known to readily cross the blood-brain barrier, exhibiting potent actions on the brain when peripherally administered.

Precision timing and dosing is paramount to the therapeutic approach. As such, the external hub can be used to determine current state of the patient. In one embodiment the relevant state is the patient's circadian phase, and with knowledge of target shift magnitude (e.g., how many time zones will the user traverse) and ideal timing of both therapies (from phase response curves), will determine the optimal dose/timing schedule, which may be initiated by the user. As such, each therapy will be administered at most once per day in one embodiment. This routine can be repeated daily, per suggestion of the system, until the entrainment is achieved. Delivery of each therapy is expected to occur within seconds of illumination of the light source, and actively regulated to a fixed level and duration by the dosing control system. Therapy production is expected to stop within about 2 minutes of turning off the light source, and presence in the blood stream is dictated by the metabolic half-life of the peptide (5-30 min). The daily timing and dosing schedule can be generated and stored in the external hub, and initiated by the user via pressing a button, voice command, etc.

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A hybrid bioelectronic implantable device containing engineered cells for delivery of therapeutic agents to a subject, the device comprising:
an implantable device implantable inside the body of the subject, wherein the implantable device comprises:
at least one cell housing containing at least one type of the engineered cells, wherein each of the engineered cells contains an optogenetic system;
an optical stimulating system within the at least one cell housing, wherein the optical stimulating system has at least one light source, wherein the optogenetic system is configured to receive a signal light from the at least one light source to control production of at least one type of therapeutic agent and a reporter agent by the engineered cells;
a permeable encapsulation material on at least a portion of a surface of the implantable device; and
an external hub disposable outside of the body of the subject, wherein in use, the external hub and the implantable device are positioned in communication via a communication method using at least one of radio frequency (RF), light, near field communication (NFC), magnetoelectric (ME), and ultrasound;
wherein in use, the at least one type of therapeutic agent is released from the cell housing into the subject's body through the permeable encapsulation.

2. The hybrid bioelectronic implantable device according to claim 1, further comprising a controller in communication with the optical stimulating system, wherein the controller is configured to control the production of the at least one type of therapeutic agent according to a control algorithm.

3. The hybrid bioelectronic implantable device according to claim 2, wherein the engineer cells are configured to start the production of the at least one type of therapeutic agent when the optogenetic systems of the engineered cells receive a signal light having a first wavelength from the optical stimulating system.

4. The hybrid bioelectronic implantable device according to claim 3, further comprising a sensing system within the at least one cell housing, sensing a fluorescent light or bioluminescence generated by the reporter agent, wherein the engineer cells are configured to stop the production of the at least one type of therapeutic agent when either the optogenetic systems of the engineered cells receive a signal light having a second wavelength from the optical stimulating system, or the sensing system detects a predetermined level of the fluorescent light or bioluminance generated by the reporter agent.

5. The hybrid bioelectronic implantable device according to claim 1, wherein a ratio of the amount of the produced reporter agent to the amount of the produced at least one type of therapeutic agent is fixed.

6. A hybrid bioelectronic implantable device containing engineered cells for delivery of therapeutic agents to a subject, the device comprising:
an implantable device implantable inside the subject's body, wherein the implantable device comprises:
at least one cell housing containing the engineered cells; and
an optical stimulating system within the at least one cell housing, wherein the optical stimulating system is configured to control production of at least one type of therapeutic agent by the engineered cells.

7. The hybrid bioelectronic implantable device according to claim 6, wherein the optical stimulating system comprises a light source to generate a light of first wavelength and a light of second wavelength different from the light of first wavelength.

8. The hybrid bioelectronic implantable device according to claim 7, wherein each of the engineered cells contains an optogenetic system configured to receive the light generated by the light source of the optical stimulating system.

9. The hybrid bioelectronic implantable device according to claim 8, wherein the engineered cells are configured to start producing the at least one type of therapeutic agent when the optogenetic systems in the engineered cells receive the light of first wavelength.

10. The hybrid bioelectronic implantable device according to claim 9, wherein the engineered cells are configured to stop producing the at least one type of therapeutic agent when the optogenetic systems in the engineered cells receive the light of second wavelength.

11. The hybrid bioelectronic implantable device according to claim 9, wherein the implantable device further comprises a sensing system disposed in the at least one cell housing, wherein the sensing system detects a signal generated by a reporter agent produced by the engineered cells, and wherein the signal comprises a biochemical signal.

12. The hybrid bioelectronic implantable device according to claim 11, wherein the engineered cells stop producing the at least one type of therapeutic agent when the signal detected by the sensing system reaches to a predetermined level.

13. The hybrid bioelectronic implantable device according to claim 11, wherein the sensing system comprises a photodiode.

14. The hybrid bioelectronic implantable device according to claim 11, wherein the reporter agent and the at least one type of therapeutic agent are produced at a fix ratio.

15. The hybrid bioelectronic implantable device according to claim 6, wherein the implantable device further comprises a permeable encapsulation material on at least a portion of its surface to allow the at least one type of therapeutic agent to be released into the subject's body through the permeable encapsulation.

16. The hybrid bioelectronic implantable device according to claim 15, wherein the permeable encapsulation material comprises a multi-layer membrane.

17. The hybrid bioelectronic implantable device according to claim 16, wherein the multi-layer membrane comprises a first layer having sub-micron pores configured to prevent immune cells of the subject from passing through the multi-layer membrane, and a second layer having micron-sized pores configured to enhance vascularization.

18. The hybrid bioelectronic implantable device according to claim 6, wherein in use, the implantable device is wirelessly coupled to an external hub disposed outside of the subject's body.

19. The hybrid bioelectronic implantable device according to claim 18, wherein the hybrid bioelectronic implantable device and the external hub are in communication with each other.

20. The hybrid bioelectronic implantable device according to claim 19, wherein the external hub is configured to collect at least one external parameter or biometric parameter, and wherein the external parameter or biometric parameter comprises an environment temperature, a location of the subject, a body temperature, a blood pressure, a heart rate, and a speed of the subject.

21. The hybrid bioelectronic implantable device according to claim 11, further comprising:
a control unit in communication with the stimulating system and the sensing system to control the stimulating system and the sensing system; and
a memory unit in communication with the control unit.

22. The hybrid bioelectronic implantable device according to claim 21, wherein the memory unit is configured to store a control algorithm to regulate production of the at least one type of therapeutic agent.

23. The hybrid bioelectronic implantable device according to claim 21, wherein the control unit and memory unit locate in the external hub.

24. The hybrid bioelectronic implantable device according to claim 19, wherein
the communication between the hybrid bioelectronic implantable device and the external hub is via a communication method using at least one of radio frequency (RF), light, near field communication (NFC), magnetoelectric (ME), and ultrasound.

25. The hybrid bioelectronic implantable device according to claim 11, further comprising a battery that is in power communication with the external hub.

26. The hybrid bioelectronic implantable device according to claim 11, wherein the implantable device comprises an oxygen generator producing oxygen for the engineered cells.

27. The hybrid bioelectronic implantable device according to claim 6, wherein the engineered cells comprise a first type of the engineered cells producing a first type of therapeutic agent, and a second type of the engineered cells producing a second type of therapeutic agent.

28. The hybrid bioelectronic implantable device according to claim 27, wherein the at least one cell housing comprises a first cell housing and a second cell housing, and wherein the first cell housing contains the first type of the engineered cells, and the second cell housing contains the second type of the engineered cells, respectively.

29. The hybrid bioelectronic implantable device according to claim 6, wherein the implantable device is implantable subcutaneously, pericardially, intracranially, or intraperitoneally.

30. The hybrid bioelectronic implantable device according to claim 13, wherein the photodiode is enclosed in a photodiode encapsulation, wherein the photodiode encapsulation reduces the amount of the light of the first wavelength reaching the photodiode.

31. A method for delivering at least one type of therapeutic agent to a subject by a hybrid bioelectronic implantable device, the method comprising:
controlling, by a control unit, a light source of an optical stimulating system located in a first cell housing of an implantable device to produce a light of first wavelength, wherein the first cell housing contains a first type of engineered cells having an optogenetic system in each of the engineered cells;
illuminating the first type of engineered cells with the light of first wavelength for a illumination time period; and
starting production of a first type of therapeutic agent by the engineered cells when the optogenetic systems in the engineered cells receive the light of first wavelength.

32. The method for delivering therapeutic agent to a subject according to claim 31, further comprising:
controlling, by the control unit, the light source of the optical stimulating system located in the first cell housing of the implantable device to produce a light of second wavelength, wherein the second wavelength is different from the first wavelength;
illuminating the first type of engineered cells with the light of second wavelength; and
stopping the production of the first type of therapeutic agent by the engineered cells when the optogenetic systems in the engineered cells receive the light of second wavelength.

33. The method for delivering therapeutic agent to a subject according to claim 32, wherein the time interval between the production of the light of first wavelength and the production of the light of the second wavelength is controlled by the control unit according to a control algorithm.

34. The method for delivering therapeutic agent to a subject according to claim 32, wherein the production of the first type of therapeutic agent by the engineered cells last for a production time period longer than the illumination time period.

35. The method for delivering therapeutic agent to a subject according to claim 31, further comprising producing a reporting agent by the engineered cells when the optogenetic systems in the engineered cells receive the light of first wavelength.

36. The method for delivering therapeutic agent to a subject according to claim 35, wherein the reporter agent generates a signal of fluorescent light signal, bioluminescence signal, impedance signal, pigment signal, or free radical signal.

37. The method for delivering therapeutic agent to a subject according to claim 36, further comprising stopping the production of the first type of therapeutic agent by the engineered cells when the signal generated by the reporter agents detected by a sensing system reaches a predetermined level, wherein the sensing system locates inside the first cell housing.

38. The method for delivering therapeutic agent to a subject according to claim 36, wherein an ratio of the amount of the produced reporter agent to the amount of the produced at least one type of therapeutic agent is fixed.

39. The method for delivering therapeutic agent to a subject according to claim 31, wherein the implantable device comprises a second cell housing containing a second type of the engineered cells, wherein the method further comprises:
controlling, by the control unit, the optical stimulating system located in the second cell housing to produce the light of first wavelength,
illuminating the second type of the engineered cells with the light of first wavelength; and
starting production of a second type of therapeutic agent by the second type of engineered cells when the optogenetic systems in the second type of engineered cells receive the light of first wavelength.

40. The method for delivering therapeutic agent to a subject according to claim 39, wherein the first type of therapeutic agent is different from the second type of therapeutic agent.

41. The method for delivering therapeutic agent to a subject according to claim 31, wherein the hybrid bioelectronic implantable device further comprises an external hub.

42. The method for delivering therapeutic agent to a subject according to claim 41, wherein the external hub is in communication with the implantable device via a communication method using at least one of radio frequency (RF), light, near field communication (NFC), magnetoelectric (ME), and ultrasound.

43. The method for delivering therapeutic agent to a subject according to claim 41, further comprising power charging the implantable device with the external hub wirelessly.

44. The method for delivering therapeutic agent to a subject according to claim 32, further comprising power charging the implantable device with an on-board battery.

45. The hybrid bioelectronic implantable device according to claim 6, further comprising an on-board battery providng power to the implantable device.
